(19) United States
(12) Patent Application Publication
(10) Pub. No.: US 2019/0106746 A1
(43) Pub. Date: Apr. 11, 2019

(54) NEXT-GENERATION SEQUENCING TO IDENTIFY ABO BLOOD GROUP

(71) Applicant: Georgetown University, Washington, DC (US)

(72) Inventors: Carolyn K. Hurley, Bethesda, MD (US); Lihua Hou, Olney, MD (US); Jennifer Ng, Rockville, MD (US)

(21) Appl. No.: 16/085,288

(22) PCT Filed: Mar. 13, 2017
(86) PCT No.: PCT/US17/22033

§ 371 Date: Sep. 14, 2018

Related U.S. Application Data
(60) Provisional application No. 62/308,423, filed on Mar. 15, 2016.

(51) Int. Cl. C12Q 1/6881; C12Q 1/6874

(52) U.S. Cl. CPC C12Q 1/6881; C12Q 2600/172; C12Q 1/6874

(57) ABSTRACT

Provided are methods of phase-defined genotyping of both alleles of the glycosyltransferase (ABO) locus of a human subject. In certain embodiments the methods include a sequencing step using next-generation sequencing. In certain embodiments the methods include a sequencing step using sequencing-by-synthesis. In certain embodiments the methods further include the steps of comparing contiguous composite nucleotide sequences to a library of reference genomic sequences encoding a region comprising exon (6) and exon (7) of the ABO locus, and identifying individual contiguous composite nucleotide sequences as either (i) a sequence encoding a region comprising a known exon (6) and exon (7) of the ABO locus, or (ii) a sequence encoding a region comprising a novel exon (6) and/or exon (7) of the ABO locus. Also provided are kits for phase-defined genotyping of both alleles of the ABO locus of a human subject.

### RELATED APPLICATIONS

This application claims benefit of U.S. Provisional Patent Application No. 62/308,423, filed Mar. 15, 2016.

### GOVERNMENT SUPPORT

This invention was made with government support under grant number N 0014-15-1-0052 awarded by the Office of Naval Research. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 20, 2017, is named 588644_GUS-016PC_SL.txt and is 18,692 bytes in size.

### BACKGROUND OF THE INVENTION

DNA sequencing is a powerful technique for identifying allelic variation within the human leukocyte antigen (HLA) genes. As DNA sequencing has been applied to other gene systems, it has become apparent that other genes, like HLA, may also be highly polymorphic and evolving by the same mechanisms as HLA. Such a gene is that encoding the glycosyltransferases that determine the blood group antigens A, B, and O.

Today DNA sequencing of human leukocyte antigen (HLA) is commonly used for unrelated donor and umbilical cord blood selection in hematopoietic progenitor cell transplantation (HPCT) used to treat leukemia, lymphoma, or other serious diseases affecting the hematopoietic system. While HLA is the primary criterion for selecting a compatible donor, physicians also consider other factors such as donor age, cytomegalovirus status, donor gender, and ABO blood group. A recent Center for International Blood and Marrow Transplant Research publication has suggested that ABO matching between donor and recipient might be beneficial although the result did not reach statistical significance (Kollman et al., The effect of donor characteristics on survival after unrelated donor transplantation for hematologic malignancy. Blood 2015 Nov. 2 pii: blood-2015-08-663823). Thus, registries of unrelated volunteers for HPCT collect and display this information along with HLA assignments in reports of volunteers potentially matching a patient requiring a transplant. ABO typing is also used in the selection of solid organ donors and blood donors.

The concept behind next-generation sequencing (NGS) technology is similar to Sanger-based DNA sequencing—the bases of a single strand of DNA are sequentially identified from signals emitted as the strand is re-synthesized to complement a DNA template strand. NGS extends this process across millions of reactions in a massively parallel fashion, rather than being limited to a single or a few DNA fragments. This enables rapid sequencing of large stretches of DNA base pairs spanning entire genomes, with the latest instruments capable of producing hundreds of gigabases of data in a single sequencing run. In a typical application, genomic DNA (gDNA) is first fragmented into a library of small segments that can be uniformly and accurately sequenced in numerous, e.g., millions or even billions, of parallel reactions. The newly identified strings of bases, called reads, are then reassembled using a known reference genome as a scaffold (resequencing), or in the absence of a reference genome (de novo sequencing). The full set of aligned reads reveals the entire sequence of each chromosome in the gDNA sample.

### SUMMARY OF THE INVENTION

An aspect of the invention is a method of genotyping of both alleles of the glycosyltransferase gene controlling A, B, and O antigens of a subject, comprising

amplifying a sample of human genomic DNA encoding exons 6 and 7 of both alleles of the glycosyltransferase (ABO) locus, thereby forming a plurality of amplicons;

fragmenting the amplicons to give a plurality of fragments about 200 to about 800 nucleotides long;

sequencing the fragments using next-generation sequencing, thereby generating a plurality of overlapping partial nucleotide sequences;

aligning the overlapping partial nucleotide sequences to determine a contiguous composite nucleotide sequence encoding the majority of each allele of the ABO locus;

comparing the contiguous composite nucleotide sequences to a library of reference genomic sequences encoding a region comprising exon 6 and exon 7 of the ABO locus; and

identifying each contiguous composite nucleotide sequence as either (i) a sequence encoding a known region comprising exon 6 and exon 7 of the ABO locus, or (ii) a sequence encoding a novel region comprising exon 6 and/or exon 7 of the ABO locus.

An aspect of the invention is a method of genotyping of both alleles of the glycosyltransferase gene controlling A, B, and O antigens of a subject, comprising

amplifying a sample of human genomic DNA encoding exons 6 and 7 of both alleles of the glycosyltransferase (ABO) locus, thereby forming a plurality of amplicons;

fragmenting the amplicons to give a plurality of fragments about 200 to about 800 nucleotides long;

sequencing the fragments using sequencing-by-synthesis, thereby generating a plurality of overlapping partial nucleotide sequences;

aligning the overlapping partial nucleotide sequences to determine a contiguous composite nucleotide sequence encoding the majority of each allele of the ABO locus;

comparing the contiguous composite nucleotide sequences to a library of reference genomic sequences encoding a region comprising exon 6 and exon 7 of the ABO locus; and

identifying each contiguous composite nucleotide sequence as either (i) a sequence encoding a known region comprising exon 6 and exon 7 of the ABO locus, or (ii) a sequence encoding a novel region comprising exon 6 and/or exon 7 of the ABO locus.

In certain embodiments, the fragmenting is randomly fragmenting.

In certain embodiments, the fragmenting comprises acoustical shearing, i.e., sonicating.

In certain embodiments, the method is performed in a multiplex manner such that the ABO locus is co-amplified with at least one HLA locus selected from the group consisting of HLA-A, -B, -C, -DRB, -DQB1, -DPB1, -DQA1, and -DPA1.

In certain embodiments, the method is performed in a multiplex manner such that the ABO locus amplicon is included with at least one HLA locus selected from the group consisting of HLA-A, -B, -C, -DRB, -DQB1, -DPB1, -DQA1, and -DPA1, for preparation of a library for a given sample or for a given individual.

An aspect of the invention is a kit, comprising

(a) paired oligonucleotide polymerase chain reaction (PCR) amplification primers suitable for use to amplify, from a sample of human genomic DNA, DNA encoding a region comprising exon 6 and exon 7 and the intervening intron of both alleles of the glycosyltransferase (ABO) locus;

(b) paired oligonucleotide adapters, each adapter oligonucleotide comprising a nucleotide sequence complementary to at least one bridge amplification primer immobilized on a substrate; and

(c) paired sequencing primers suitable for use to sequence amplification products prepared using the paired PCR amplification primers.

In certain embodiments, the kit further comprises paired oligonucleotide PCR amplification primers suitable for use to amplify, from a sample of human genomic DNA, DNA encoding both alleles of at least one human leukocyte antigen (HLA) locus.

In certain embodiments, the kit further comprises paired oligonucleotide adapters, each adapter with a unique sequence to be used in identifying the source of an amplicon.

In certain embodiments, the kit further comprises at least one enzyme selected from the group consisting of T4 DNA polymerase, Klenow fragment of T4 DNA polymerase, and T4 polynucleotide kinase; at least one buffer suitable for activity of said T4 DNA polymerase, Klenow fragment of T4 DNA polymerase, or T4 polynucleotide kinase in repairing DNA fragments generated, for example, by acoustical shearing; and, optionally, a DNA polymerase and dATP in a buffer suitable for activity of said DNA polymerase.

In certain embodiments, the paired PCR amplification primers for the ABO locus are

(sense)
(SEQ ID NO: 1)
5′-CCCTTTGCTTTCTCTGACTTGCG-3′;
and
(antisense)
(SEQ ID NO: 2)
5′-AGTTACTCACAACAGGACGGACA-3′.

### BRIEF DESCRIPTION OF THE DRAWINGS

FIG. 1 is a schematic drawing depicting the simple inheritance of the ABO blood group.

FIG. 2 is a schematic drawing of the enzymatic activity of the ABO glycosyltransferase showing how the A and B antigens are created and the impact on glycosylation of the H antigen.

FIG. 3 is a schematic diagram depicting the ABO antigens and naturally occurring antibodies to these antigens.

FIG. 4 is a schematic drawing of the structure of an ABO glycosyltransferase protein bound to a sugar. Yellow (light shading) indicates the key residues impacting the specificity of the catalytic site. The catalytic site in the enzyme encoding the A antigen differs from that of the enzyme encoding the B antigen at amino acid residues 235, 266, and 268 (G, L, and G for glycosyltransferase A; and S, M, and A for glycosyltransferase B, respectively). B comprises the amino acid sequence GDFYYMGAFFGGS (SEQ ID NO:9), and A comprises the amino acid sequence GDFYYLGGFFGGS (SEQ ID NO:10).

FIG. 5 is a schematic drawing of the DNA exon and intron structure of the ABO gene showing the position of the amplicon used for DNA sequencing.

FIG. 6 is an example of Sequencher (Gene Codes Corp.) software output showing the nucleotide sequence of the reads in the region of exon 7 that includes a deletion that creates the alleles encoding a subgroup of A called A2 Amino acid sequence LRCPRTTRRSGTRERLPGALGGLPAAPSPSRPWF corresponds to SEQ ID NO:11. Nucleotide sequence CTGCGGTGCCCAAGAACCACCAGGCGGTCCGGAACCCGTG AGCGGCTGCCAGGGGCTCTGGGAGGGCTGCCAGCAGCCCCGTCCCCCTCCCGC CCTTGGTTTT corresponds to SEQ ID NO:12. Nucleotide sequence CTGCGGTGCCCA AGAACCACCAGGCGGTCCGGAA*CCGTGAGCGGCTGCCAGGGGCTCTGGGAGG GCTGCCAGCAGCCCCGTCCCCCTCCCGCCCTTGGTTTT corresponds to SEQ ID NO:13. Nucleotide sequence CTGCGGTGCCAAAGAACCACCAGGCGGTCCGGAA* CCGTGAGCGGCTGCCAGGGGCTCTGGGAGGGCTGCCAGCAGCCCCGTCCCCCT CCCGCCCTTGGTTTT corresponds to SEQ ID NO:14. Nucleotide sequence CTGCGGT GCCCAAGAACCACCAGGCGGCCCGGAACCCGTGAGCGGCTGCCAGGGGCTCTG GGAGGGCTGCCAGCAGCCCCGT*CCCCTCCCGCCCTTGGTTTT corresponds to SEQ ID NO:15. Nucleotide sequence CTGCGGTGCCCAAGAGCCACCAGGCGGTCC GGAA*CCGTGAGCGGCTGCCAGGGGCTCTGGGAGGGCTGCCAGCAGCCCCGTC CCCCTCCCGCCCTTGGTTTT corresponds to SEQ ID NO:16. Nucleotide sequence CTGCGGTGCCCAAGAACCACCAGGCGGTCCGG*ACCCGTGAGCGGCTGCCAGG GGCTCTGGGAGGGCTGCCAGCAGCCCCGTCCCCCTCCCGCCCTTGGTTTT corresponds to SEQ ID NO:17. Nucleotide sequence CTGCGGTGCCCAAGAACCACC AGGCGGTCTGGAA*CCGTGAGCGGCTGCCAGGGGCTCTGGGAGGGCTGCCAGC AGCCCCGTCCCCCTCCCGCCCTTGGTTTT corresponds to SEQ ID NO: 18. Nucleotide sequence CTGCGGTGCCCAAGAACCACCAGGCGGTCCGGAACCCGTG AGCGGCTGCCAGGGGCTCTGGGAGGGCTGCCAGCAG*CCCGTCCCCCTCCCGCC CTTGGTTTT corresponds to SEQ ID NO:19. Nucleotide sequence CTTCGGTGCCCAA GAACCACCAGGCGGTCCGGAA*CCGTAAGCGGCTGCCAGGGGCTCTGGGAGGG CTGCCAGCAGCCCCGTCCCCCTCCCGCCCTTGGTTTT corresponds to SEQ ID NO:20. Nucleotide sequence CTGCGGTGCCCAAGAACCACCAGGCGGTCCGGAA*C CGCGAGCGGCTGCCAGGGGCTCTGGGAGGGCTGCCAGCAGCCTCGTCCCCCTC CCGCCCTTGGTTTT corresponds to SEQ ID NO:21. Nucleotide sequence CTGCGGTG CCCAAGAACCCCCAGGCGGTCCGGAA*CCGTGAGCGGCTGCCAGGGGCTCTGG GAGGGCTGCCAGCAGCCCCGGCCCCCTCCCGCCCTTGGTTTT corresponds to SEQ ID NO:22. Nucleotide sequence CTGCGGTGCCCAAGAACCACCAGGCGGTCCGGAA *CCGTGAGCGGCTGCCAGGGGCTCTGGGATGGCTGCCAGCAGCCCCGTCCCCCT CCCGCCCTTTGTTTT corresponds to SEQ ID NO:23.

FIG. 7 depicts ABO nucleotide sequence analysis using Connexio Assign MPS software. The figure shows a map of a region containing exons 6 and 7 and assigns the genotype, A*101+A*101 on the right of the figure. Shown are nucleotide sequences CTCGTGGTGACCCCTTGGCTGGCTCCCATTGTCTGGGAGGGCACRTTCAACATC GACATCCTCAAYGAGCAGTTCAGGCTCCAGAACACCACCATTG (SEQ ID NO:24) and CTCGTGGTGACCCCTTGGCTGGCTCCCATTGTCTGGGAGGGCACTTTCAAC ATCGACATCCTCAACGAGCAGTTCAGGCTCCAGAACACCACCATTG (SEQ ID NO:25).

FIGS. 8A and 8B depict certain subsequences, from two samples, determined in accordance with the invention. Nucleotide 612 in this NGS ABO subsequence is a deletion in many of the 0 alleles. FIG. 8A shows a heterozygous position with a G (black bar) and a single nucleotide deletion (gray bar) at position 612; this sample types as A+O. Nucleotide sequence CTCGTGGTGACCCCTTGG corresponds to SEQ ID NO:26, and nucleotide sequence CTCGTGGT-ACCCCTTGG corresponds to SEQ ID NO:27. FIG. 8B shows a homozygous deletion at position 612 and the genotype assigned is O*02+O*02. Nucleotide sequence GTGGTGACCC corresponds to SEQ ID NO:28, and nucleotide sequence GTGGT-ACCC corresponds to SEQ ID NO:29.

FIG. 9 depicts nucleotide position 2464 at the 3′ end of exon 7. There is a C deletion at this position in the A subgroup called A2. In this figure the position is heterozygous: one allele (O*01) has a C, and the second allele (A2*1012) has a deletion. The alignment from the Connexio Assign MPS software shows a short read lower down in the figure that is caused by the frameshift. Nucleotide sequence GGAACCSKKRAGCG corresponds to SEQ ID NO:30, and nucleotide sequence GGAACCCGTGAGCG corresponds to SEQ ID NO:31.

FIG. 10 depicts Connexio Assign analysis program for the catalytic site-encoding region of the sequence of a sample typed as A*101+B*101. Within the DNA sequence that encodes the catalytic site of the ABO transferase, A and B alleles differ in nucleotide sequence (A has the sequence CCTGGGGGGGT (SEQ ID NO:3), and B has the sequence CATGGGGGCGT (SEQ ID NO:4)). Nucleotide sequence TACMTGGGGRSGTTC corresponds to SEQ ID NO:32; nucleotide sequence TACCTGGGGRGGTTC corresponds to SEQ ID NO:33; nucleotide sequence TACATGGGGRCGTTC corresponds to SEQ ID NO: 34; and nucleotide sequence TACMTGGGGGSGTTC corresponds to SEQ ID NO:35.

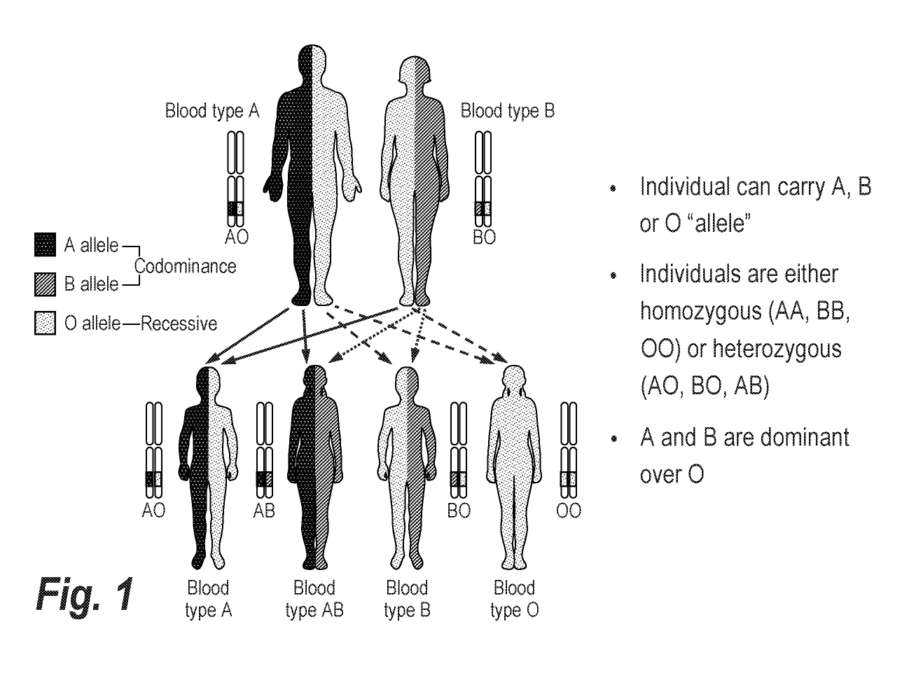

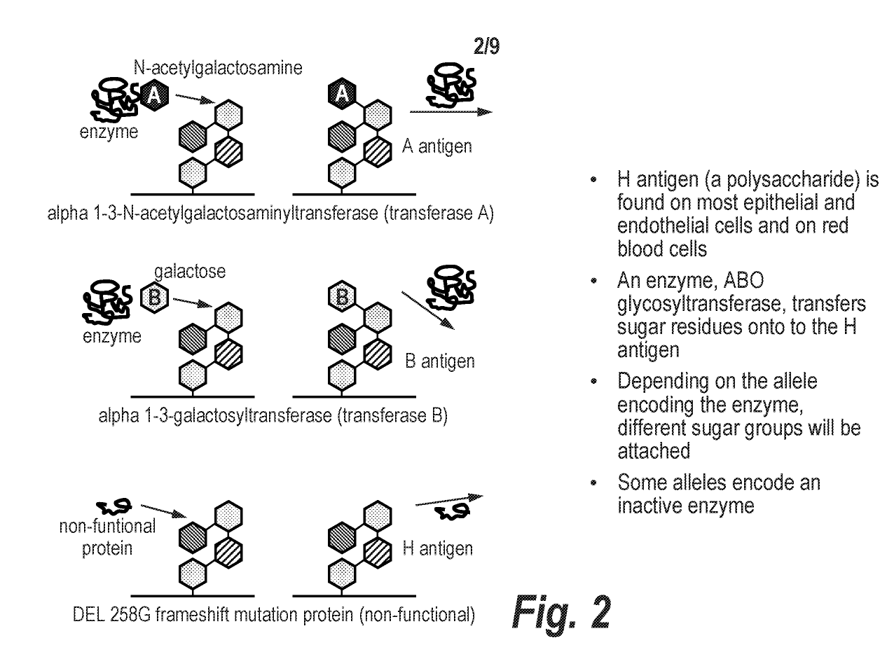

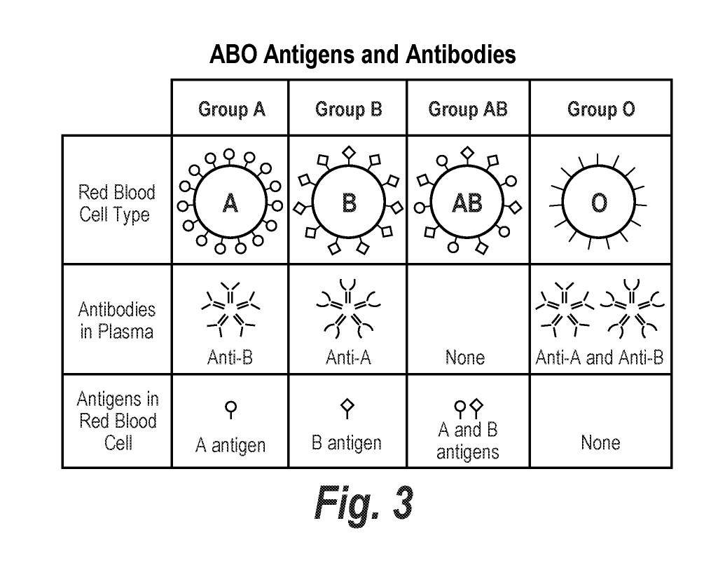

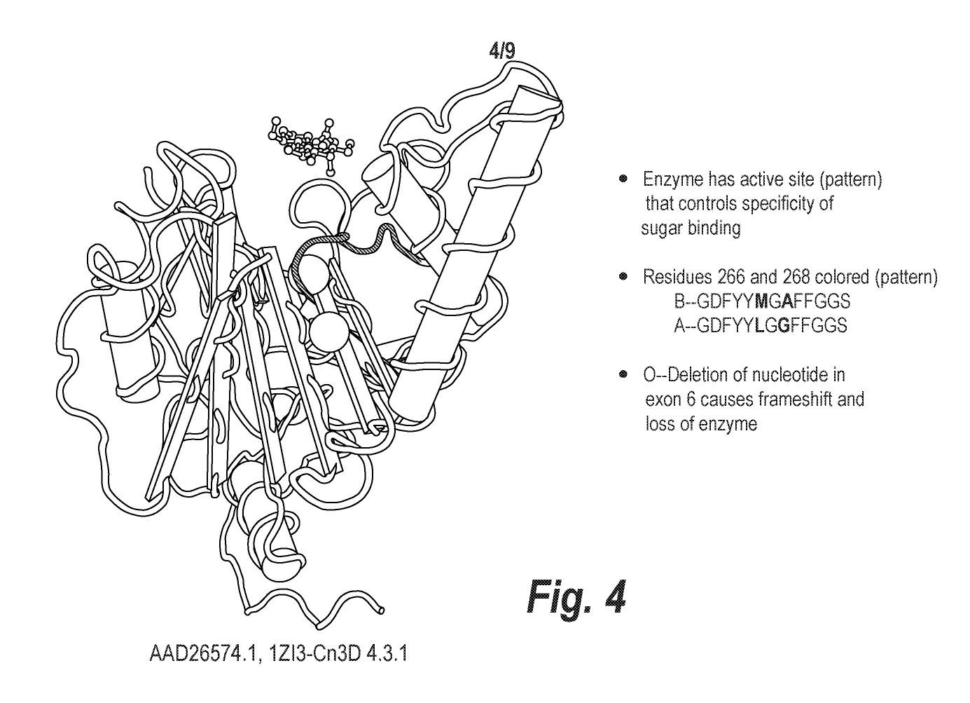

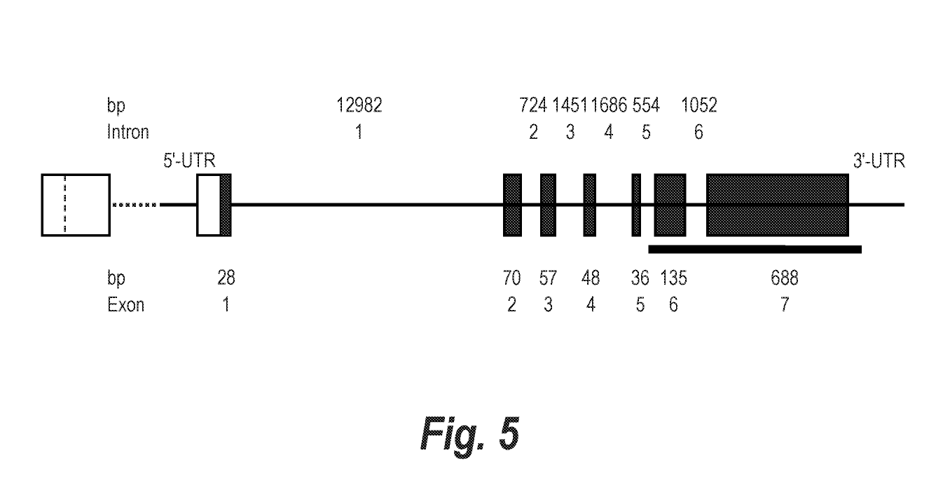

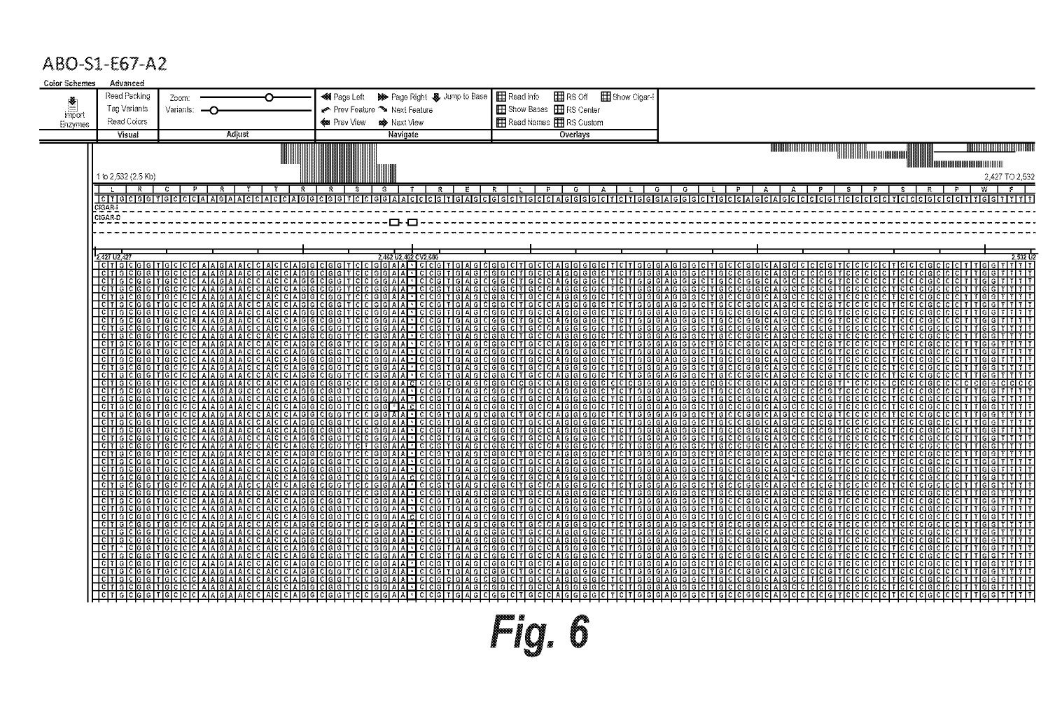

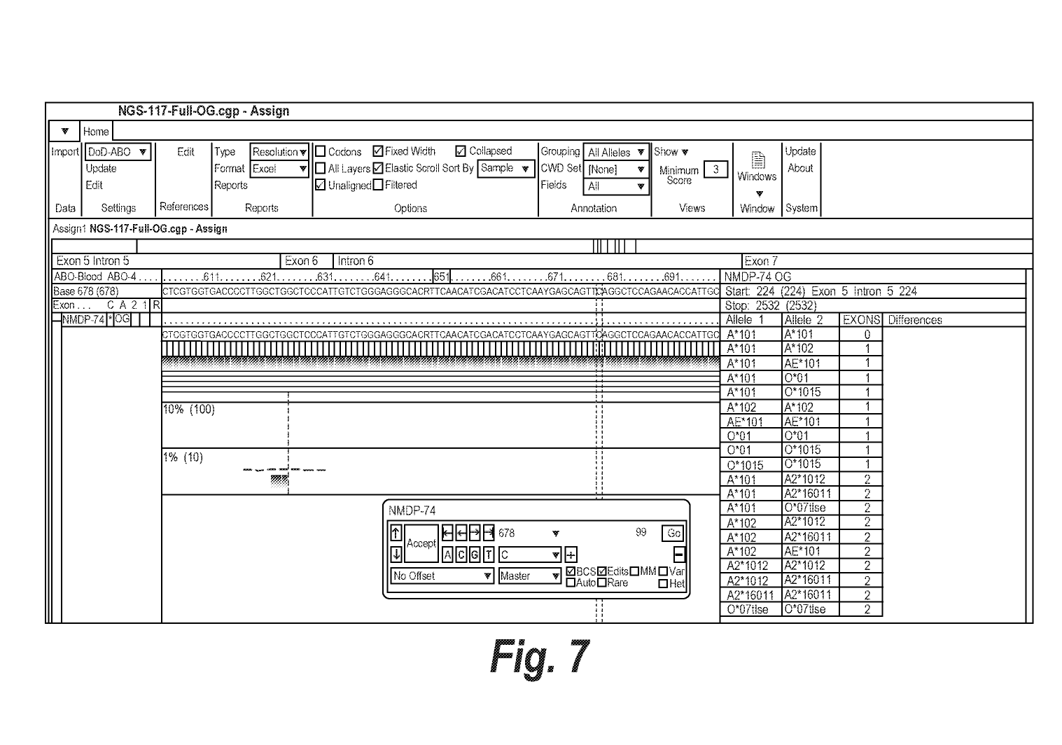

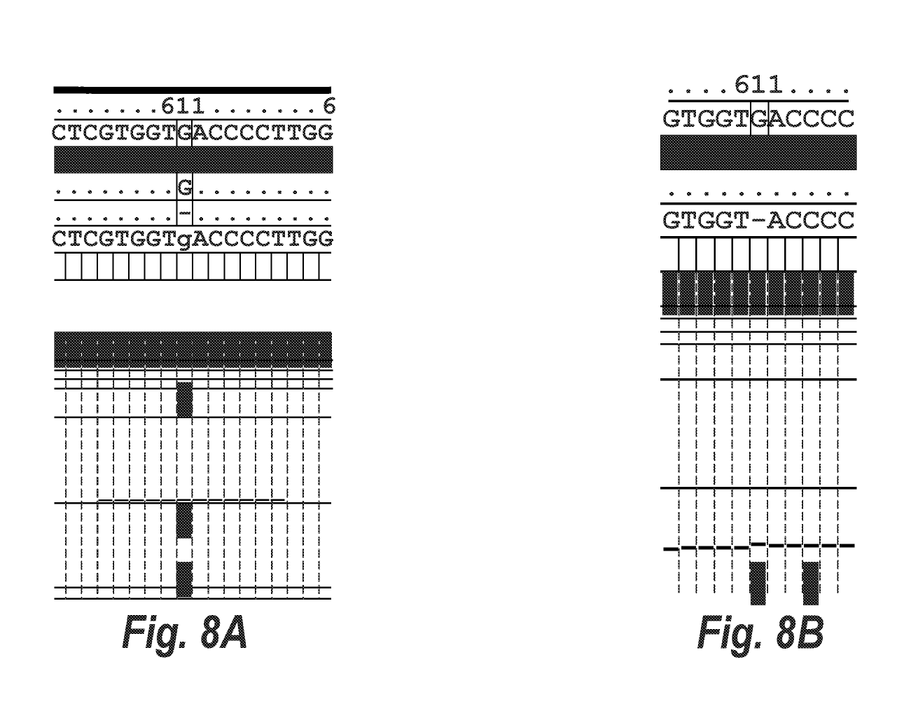

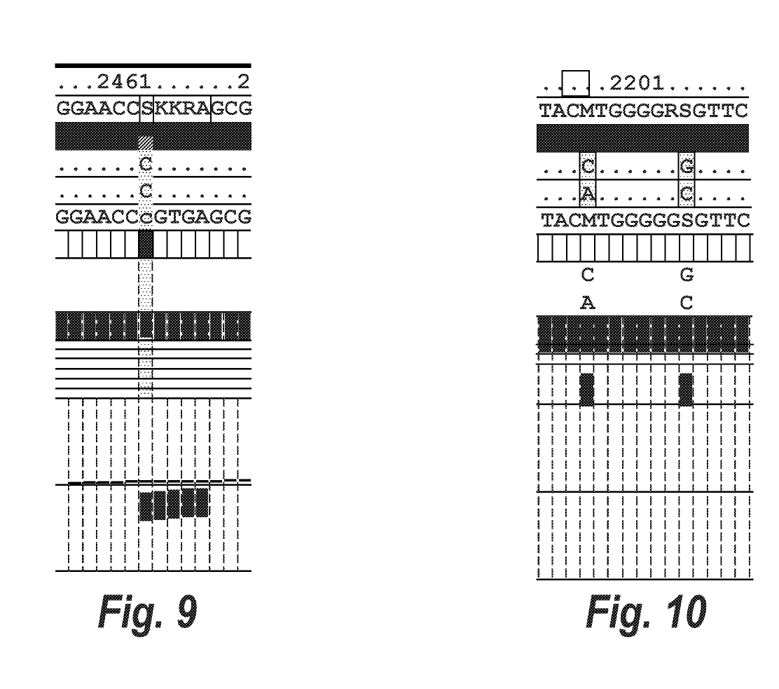

### DETAILED DESCRIPTION OF THE INVENTION

One challenge for unrelated hematopoietic progenitor cell donor registries is how to identify the factors important in the selection of unrelated donors in a rapid and cost-effective manner when initially listing the volunteer on the registry. The first priority is to obtain human leukocyte antigen (HLA) assignments for a minimal of four loci, HLA-A, HLA-B, HLA-C, and HLA-DRB1. The continuing discovery of novel alleles has resulted in loci with hundreds to thousands of alleles, for example, HLA-B with over 3000 alleles. DNA-based typing results obtained at recruitment of registry volunteers usually include many alternative (or ambiguous) genotypes. An added complexity for typing is that more than one pair of alleles share a diploid DNA sequence for these exons. These pairs of alleles differ in the phase of the polymorphisms, i.e., which of the alternative polymorphic nucleotides are located on a specific homologue of chromosome 6. As novel alleles are identified, the number of pairs of alleles sharing a diploid sequence increases and new ambiguities are identified.

This means that, because of cost constraints, additional testing is required prior to donor selection to “phase” polymorphic nucleotides. This slows down the process and would not be ideal in a contingency situation. Even more important, however, is the impact of secondary assays on the robust nature of the HLA assignment. Today primary data and test reagents used in secondary assays are not readily incorporated into the initial result and are not captured by the registry. This is particularly true if the secondary assay uses a testing technology different from the initial assay (e.g., DNA sequencing followed by sequence-specific priming) In these cases, laboratory software is unlikely to capture and merge primary data from both results, making it difficult for the registry to collect this information. A second limitation is that the reagents used are selected based on the current alternative genotypes and do not take into account new alternatives that will appear over time. Next-generation sequencing of many volunteers at the time of recruitment provides an advantage in that single molecules of DNA are sequenced so that alleles are routinely separated and ambiguity is reduced. This should allow more rapid donor selection.

At the same time of HLA typing, it would be cost-effective to test for other genes that play a role in donor selection. An advantage of this invention is that the gene encoding the blood group A, B, O antigens can be included within the next-generation sequencing assay.

There are a number of methods of NGS, including sequencing-by-synthesis, single-molecule real-time sequencing, ion semiconductor sequencing, pyrosequencing, and sequencing by ligation. The sequencing-by-synthesis method was developed by Shankar Balasubramanian and David Klenerman at the University of Cambridge, and it is described in International Publication No. WO 00/06770 and U.S. Pat. Nos. 6,787,308 and 7,232,656, the entire disclosures of which are incorporated herein by reference.

Methods and compounds for use in NGS for phased HLA Class I and Class II antigens are disclosed in PCT Patent Application No. PCT/US2015/053087, the entire content of which is incorporated herein by reference.

### ABO Antigens

The ABO blood system is the primary antigen system important in blood transfusion and solid organ transplantation. Recent evidence suggests that it might also be important in hematopoietic progenitor cell transplantation. This blood system is controlled by the activity of a glycosyltransferase (GTA or GTB) that attaches sugar residues (either N-acetylgalactosamine or galactose) to a common substrate (the H antigen). The enzyme has several phenotypic variants which either alter the carbohydrate attached (N-acetylgalactosamine (A) vs galactose (B)) or cause loss of expression of the enzyme so the H antigen is not modified (0). A variant of A, A2, has a reduced level of N-acetylgalactosamine addition. These variants are discriminated currently by serology and by lectin binding (defining A1 vs A2). Serology can either detect the modification of the H antigen or can detect the presence of naturally-occurring antibodies directed to A and/or B (e.g., a person with the B pattern of glycosylation will have antibodies directed to A).

In humans the glycosyltransferase locus, equivalently referred to herein as the ABO locus or the ABO glycosyltransferase locus, is located on chromosome 9 and contains seven exons that span more than 18 kb of genomic DNA. Exon 7 is the largest and contains most of the coding sequence. The ABO locus has three main alleleic forms: A, B, and O. The A “allele” (also referred to as A1 or A2) encodes a glycosyltransferase that bonds α-N-acetylgalactosamine to the D-galactose end of the H antigen, producing the A antigen. The B allele encodes a glycosyltransferase that bonds α-D-galactose to the D-galactose end of the H antigen, creating the B antigen. The O allele encodes a nonfunctional form of glycosyltransferase, resulting in an unmodified H antigen, creating the O phenotype.

On the genomic level, the glycosyltransferase gene has many alleles (˜300). Table 1 below lists some of the more common sequence variants that, in general, differentiate among A, B, and O, and between the subgroups A1 and A2, and that control the activity and specificity of the enzyme. The sequence encoding the catalytic site of the enzyme lies in exon 7 of the gene; key amino acid residues 235, 266, and 268 control the specificity of this active site. Furthermore, a common nucleotide deletion in exon 6 creates a stop codon that abolishes synthesis of full-length glycosyltransferase, leading to the O or null phenotype. In accordance with the present invention, the amplicon includes both of these exons (exons 6 and 7), the intervening intron (intron 6), and portions of intron 5 and the 3′-UTR (3′-untranslated region).

TABLE 1

| | Literature nucleotide numbering | | | | |
|---|---|---|---|---|---|
| | 261 | 703 | 796 | 803 | 1061 |
| | Literature amino acid numbering | | | | |
| | | 235 | 266 | 268 | |
| A (A1) | | Gly | Leu | Gly | |
| A (A2) | | Gly | Leu | Gly | Del adds 21 |
| | | | | | amino acids |
| B | | Ser | Met | Ala | |
| O | Del truncates | | | | |
| | protein | | | | |
| O w/o deletion | | Gly | Leu | Arg | |

There are also variants that give “unusual” serologic typing patterns, for example, weak A (i.e., weaker than A2) or weak B results. These are infrequent and usually result from unique sequence variations that alter the enzyme activity or specificity. O variants without the usual deletion in exon 6 also result from deletions in other regions of the gene or alterations that inactivate the enzyme's catalytic site (e.g., last entry in Table 1).

### ABO Genotyping

ABO phenotypes are used in solid organ and hematopoietic progenitor cell transplantation for donor selection and in blood transfusion to reduce immune responses to foreign tissue. Individuals have naturally occurring antibodies to the A and B antigens depending on their ABO genotype (e.g., type A individuals have antibodies directed to the B antigen) and these antibodies can cause transfusion reactions and graft rejection. The instant invention allows the rapid and accurate determination of the sequence and phase of nucleotide polymorphisms in the functionally important region of the ABO glycosyltransferase protein, i.e., which polymorphic nucleotides are encoded by the same allele. This allows the assignment of ABO at the time of HLA genotyping, and thereby provides more complete information for use in donor selection for matching for transplantation and transfusion.

In accordance with the instant invention, the nucleotide sequence and the phase of polymorphic nucleotides in the functionally relevant region of the genomic or other DNA encoding ABO glycosyltransferase is determined in a single next-generation sequence run at the time of initial HLA typing.

In accordance with the instant invention, polymerase chain reaction (PCR) amplicons including exons encoding the key functional regions of the ABO glycosyltransferase and intervening intron (and, optionally, flanking intron and/or untranslated sequence) are sequenced using next-generation sequencing. In certain embodiments, polymerase chain reaction (PCR) amplicons including exons encoding the key functional regions of the ABO glycosyltransferase and intervening intron (and, optionally, flanking intron and/or untranslated sequence) are sequenced using sequencing-by-synthesis technique. By including the intron in next-generation sequencing, the phase of polymorphic residues is established throughout exons 6 and 7. This allows the identification of the genotypes present or absent without ambiguity.

Advantageously, the methods of the invention can be used in multiplex format to process, simultaneously, genomic DNA from a plurality of unique samples, e.g., genomic DNA from multiple individuals. A further advantage is the ability to combine two analytes, ABO and HLA, into one assay.

Targeted resequencing employed by the methods of the present invention focuses on the PCR-amplified ABO glycosyltransferase gene with amplification of a region of the ABO glycosyltransferase gene encoding the majority of the glycosyltransferase protein including the catalytic site-encoding exons and intervening intron. Until now, only Sanger sequencing has been employed to characterize this gene in situations where classical serology suggests unique phenotypes. Such Sanger sequencing does not permit phasing of polymorphic residues to establish single genotypes and a single analysis of multiple amplicon sequences.

In certain embodiments, the methods of the invention include, in a general sense, the steps of amplifying genomic DNA; fragmenting the amplified DNA; attaching bar codes and annealing sites (sequencing adapters), for example through a second round of PCR; PCR clean-up and size selection; sample normalization and pooling of multiple samples to form a library; sequencing by synthesis, for example using an Illumina® (San Diego, Calif.) platform; and analyzing sequence data.

### Sequencing-By-Synthesis

The sequencing-by-synthesis method is similar to Sanger sequencing, but it uses modified dNTPs containing a terminator which blocks further polymerization, so only a single base can be added by a polymerase enzyme to each growing DNA copy strand. The sequencing reaction is conducted simultaneously on a very large number (many millions or more) of different template molecules spread out on a solid surface, e.g., a surface of a flow cell. The terminator also contains a fluorescent label, which can be detected by a camera or other suitable optical device.

In a common embodiment, sequencing-by-synthesis technology uses four fluorescently labeled nucleotides to sequence the tens of millions of clusters on the flow cell surface in parallel. During each sequencing cycle, a single labeled deoxynucleoside triphosphate (dNTP) is added to the nucleic acid chain. The nucleotide label serves as a terminator for polymerization, so after each dNTP incorporation, the fluorescent dye is imaged to identify the base and then enzymatically cleaved to allow incorporation of the next nucleotide. Since all four reversible terminator-bound dNTPs (A, C, T, G) are present as single, separate molecules, natural competition minimizes incorporation bias. Base calls are made directly from signal intensity measurements during each cycle, which greatly reduces raw error rates compared to other technologies. The end result is highly accurate base-by-base sequencing that eliminates sequence-context specific errors, enabling robust base calling across the genome, including repetitive sequence regions and within homopolymers.

In an alternative embodiment, only a single fluorescent color is used, so each of the four bases must be added in a separate cycle of DNA synthesis and imaging. Following the addition of the four dNTPs to the templates, the images are recorded and the terminators are removed. This chemistry is called “reversible terminators”. Finally, another four cycles of dNTP additions are initiated. Since single bases are added to all templates in a uniform fashion, the sequencing process produces a set of DNA sequence reads of uniform length.

Although the fluorescent imaging system used in sequencers is not sensitive enough to detect the signal from a single template molecule, the major innovation of the sequencing-by-synthesis method is the amplification of template molecules on a solid surface. The DNA sample is prepared into a “sequencing library” by the fragmentation into pieces each typically around 200 to 800 nucleotides long. Custom adapters are added to each end and the library is flowed across a solid surface (the “flow cell”), whereby the template fragments bind to this surface. Following this, a solid phase “bridge amplification” PCR process (cluster generation) creates approximately one million copies of each template in tight physical clusters on the flow cell surface. These clusters are of sufficient size and density to permit signal detection.

Amplicon sequencing allows researchers to sequence small, selected regions of the genome spanning hundreds of base pairs. Commercially available NGS amplicon library preparation kits allow researchers to perform rapid in-solution amplification of custom-targeted regions from genomic DNA. Using this approach, thousands of amplicons spanning multiple samples can be simultaneously prepared and indexed in a matter of hours. With the ability to process numerous amplicons and samples on a single run, NGS is much more cost-effective than CE (capillary electrophoresis)-based Sanger sequencing technology, which does not scale with the number of regions and samples required in complex study designs. NGS enables researchers to simultaneously analyze all genomic content of interest from multiple individuals in a single experiment, at a fraction of the time and cost.

This highly targeted NGS approach enables a wide range of applications for discovering, validating, and screening genetic variants for various study objectives. Amplicon sequencing is well-suited for clinical environments, where researchers are examining a limited number of treatment-related highly polymorphic genes like ABO glycosyltransferase.

### Methods of the Invention

An aspect of the invention is a method of phase-defined genotyping of both alleles of the glycosyltransferase (ABO) locus of a subject, comprising

amplifying a sample of human genomic DNA encoding a region comprising exon 6 and exon 7 of both alleles of the ABO locus, thereby forming a plurality of amplicons;

fragmenting the amplicons to give a plurality of fragments of about 200 to about 800 nucleotides long;

sequencing the fragments using next-generation sequencing, thereby generating a plurality of overlapping partial nucleotide sequences;

aligning the overlapping partial nucleotide sequences to determine a contiguous composite nucleotide sequence encoding a region comprising exon 6 and exon 7 of each allele of the ABO locus; and

comparing the contiguous composite nucleotide sequences to a library of reference genomic sequences encoding a region comprising exon 6 and exon 7 of the ABO locus.

In certain embodiments, the comparing step comprises comparing the contiguous composite nucleotide sequences to a library of reference genomic and cDNA sequences encoding a region comprising exon 6 and exon 7 of the ABO locus.

In certain embodiments, the method further includes the step of identifying each contiguous composite nucleotide sequence as either (i) a sequence encoding a region comprising a known exon 6 and exon 7 of the ABO locus, or (ii) a sequence encoding a region comprising a novel exon 6 and/or exon 7 of the ABO locus.

An aspect of the invention is a method of phase-defined genotyping of both alleles of the glycosyltransferase (ABO) locus of a subject, comprising

amplifying a sample of human genomic DNA encoding a region comprising exon 6 and exon 7 of both alleles of the ABO locus, thereby forming a plurality of amplicons;

fragmenting the amplicons to give a plurality of fragments of about 200 to about 800 nucleotides long;

sequencing the fragments using sequencing-by-synthesis, thereby generating a plurality of overlapping partial nucleotide sequences;

aligning the overlapping partial nucleotide sequences to determine a contiguous composite nucleotide sequence encoding a region comprising exon 6 and exon 7 of each allele of the ABO locus; and

comparing the contiguous composite nucleotide sequences to a library of reference genomic sequences encoding a region comprising exon 6 and exon 7 of the ABO locus.

In certain embodiments, the comparing step comprises comparing the contiguous composite nucleotide sequences to a library of reference genomic and cDNA sequences encoding a region comprising exon 6 and exon 7 of the ABO locus.

In certain embodiments, the method further includes the step of identifying each contiguous composite nucleotide sequence as either (i) a sequence encoding a region comprising a known exon 6 and exon 7 of the ABO locus, or (ii) a sequence encoding a region comprising a novel exon 6 and/or exon 7 of the ABO locus.

As discussed above, there are numerous ABO proteins encoded by a single locus. Normally, each nucleated diploid cell has both a maternal allele and a paternal allele for each ABO locus, e.g., a maternal ABO allele and a paternal ABO allele. In accordance with the methods of the invention, not only can both alleles of ABO locus be sequenced and phased simultaneously, but also both alleles of a plurality of loci can be sequenced and phased simultaneously. For example, the plurality of loci to be sequenced and phased simultaneously can be obtained from a plurality of subjects.

Similar to the ABO locus, there are numerous HLA proteins encoded by a single locus. Normally, each nucleated diploid cell has both a maternal allele and a paternal allele for each HLA locus, e.g., a maternal HLA-A allele and a paternal HLA-A allele. In accordance with the methods of the invention, not only can both alleles of a given HLA locus be sequenced and phased simultaneously, but also both alleles of a plurality of loci can be sequenced and phased simultaneously. For example, the plurality of loci to be sequenced and phased simultaneously can be obtained from a plurality of subjects.

In certain embodiments, both alleles of the ABO locus of a subject are phase-defined.

In certain embodiments, both alleles of the ABO locus and both alleles of at least one HLA class I locus of a subject are phase-defined.

In certain embodiments, both alleles of the ABO locus and both alleles of at least one HLA class II locus of a subject are phase-defined.

In certain embodiments, both alleles of the ABO locus, both alleles of at least one HLA class I locus, and both alleles of at least one HLA class II locus of a subject are phase-defined.

In certain embodiments, the at least one HLA class I locus is HLA-A.

In certain embodiments, the at least one HLA class I locus is HLA-B.

In certain embodiments, the at least one HLA class I locus is HLA-C.

In certain embodiments, the at least one HLA class I locus is HLA-A and HLA-B.

In certain embodiments, the at least one HLA class I locus is HLA-A and HLA-C.

In certain embodiments, the at least one HLA class I locus is HLA-B and HLA-C.

In certain embodiments, the at least one HLA locus is HLA-A, HLA-B, and HLA-C.

In certain embodiments, the at least one HLA class II locus is HLA-DRB.

In certain embodiments, the at least one HLA class II locus is HLA-DQB1.

In certain embodiments, the at least one HLA class II locus is HLA-DPB1.

In certain embodiments, the at least one HLA class II locus is HLA-DQA1.

In certain embodiments, the at least one HLA class II locus is HLA-DPA1.

In certain embodiments, the at least one HLA class II locus is HLA-DRB and HLA-DQB 1.

In certain embodiments, the at least one HLA class II locus is HLA-DRB and HLA-DPB1.

In certain embodiments, the at least one HLA class II locus is HLA-DRB and HLA-DQA1.

In certain embodiments, the at least one HLA class II locus is HLA-DRB and HLA-DPA 1.

In certain embodiments, the at least one HLA class II locus is HLA-DQB1 and HLA-DPB1.

In certain embodiments, the at least one HLA class II locus is HLA-DQB1 and HLA-DQA1.

In certain embodiments, the at least one HLA class II locus is HLA-DQB1 and HLA-DPA1.

In certain embodiments, the at least one HLA class II locus is HLA-DPB1 and HLA-DQA1.

In certain embodiments, the at least one HLA class II locus is HLA-DPB1 and HLA-DPA1.

In certain embodiments, the at least one HLA class II locus is HLA-DQA1 and HLA-DPA1.

In certain embodiments, the at least one HLA class II locus is HLA-DRB, HLA-DQB 1, and HLA-DPB 1.

In certain embodiments, the at least one HLA class II locus is HLA-DRB, HLA-DPB 1, and HLA-DQA 1.

In certain embodiments, the at least one HLA class II locus is HLA-DRB, HLA-DQA 1 , and HLA-DPA 1.

In certain embodiments, the at least one HLA class II locus is HLA-DRB, HLA-DQB 1 , and HLA-DQA1.

In certain embodiments, the at least one HLA class II locus is HLA-DRB, HLA-DQB 1, and HLA-DPA 1.

In certain embodiments, the at least one HLA class II locus is HLA-DRB, HLA-DPB 1, and HLA-DPA1.

In certain embodiments, the at least one HLA class II locus is HLA-DQB1, HLA-DPB 1, and HLA-DQA 1.

In certain embodiments, the at least one HLA class II locus is HLA-DQB1, HLA-DPB 1, and HLA-DPA1.

In certain embodiments, the at least one HLA class II locus is HLA-DPB1, HLA-DQA 1, and HLA-DPA 1.

In certain embodiments, the at least one HLA class II locus is HLA-DQB1, HLA-DQA 1, and HLA-DPA 1.

In certain embodiments, the at least one HLA class II locus is HLA-DRB, HLA-DQB 1, HLA-DPB 1 , and HLA-DQA1.

In certain embodiments, the at least one HLA class II locus is HLA-DRB, HLA-DQB 1, HLA-DPB 1, and HLA-DPA 1.

In certain embodiments, the at least one HLA class II locus is HLA-DRB, HLA-DQB 1 , HLA-DQA 1, and HLA-DPA 1.

In certain embodiments, the at least one HLA class II locus is HLA-DRB, HLA-DPB 1, HLA-DQA 1, and HLA-DPA 1.

In certain embodiments, the at least one HLA class II locus is HLA-DQB1, HLA-DPB 1, HLA-DQA 1, and HLA-DPA 1.

In certain embodiments, the at least one HLA class II locus is HLA-DRB, HLA-DQB 1, HLA-DPB 1, HLA-DQA 1 , and HLA-DPA 1.

The term “phase-defined genotyping” as used herein generally refers to elucidating the nucleotide sequence of a single allele of any given locus on a first chromosome with sufficient detail to distinguish it from a heterologous allele at the same locus on a second chromosome. In certain embodiments, the term “phase-defined genotyping” as used herein refers to elucidating the nucleotide sequence of a single allele of an ABO-encoding locus on a first chromosome with sufficient detail to distinguish it from a heterologous allele at the same locus on a second chromosome. In a preferred embodiment, “phase-defined genotyping” refers to elucidating the nucleotide sequences of both alleles of an ABO-encoding locus with sufficient detail to distinguish one allele from the other and one genotype from another. Of course, when two alleles (e.g., maternal and paternal alleles) are completely identical, it will not be possible to distinguish one from the other. Information generated by the method is used to separate two chromosomes and to determine the two phase-defined ABO gene sequences for the ABO locus of a subject. Taking advantage of highly polymorphic nature of the ABO gene, wide-ranged library size, and massive parallel sequencing, it becomes possible to phase sequence reads on a chromosome and tile phased reads to generate ABO gene sequences to accompany the HLA gene sequences from large numbers of individuals needed to maintain a hematopoietic progenitor cell registry of volunteer donors.

In certain embodiments, the term “phase-defined genotyping” as used herein refers to elucidating the nucleotide sequence of a single allele of an ABO-encoding locus on a first chromosome with sufficient detail to distinguish it from a reference allele at the same locus on a second chromosome. In such embodiments the reference allele can be a known haplotype sequence, for example, a haplotype sequence in a library of known haplotype sequences.

Amplification primers have been reported by Chen et al. (ABO sequence analysis in an AB type with anti-B patient. Chinese Medical Journal 2014; 127:971-2). The primers were selected so that, when they are used to amplify a sample of human genomic DNA encoding a region comprising exons 6 and 7 of both alleles of the ABO locus, the resulting amplification products include a plurality of amplicons comprising sequence encoding the majority of both alleles of the ABO locus.

For ABO, DNA encoding the majority of the protein and the catalytic site generally includes all of exon 6, all of intron 6, and all of exon 7. Accordingly, in certain embodiments, each amplicon comprises DNA encoding all of exon 6, all of intron 6, and all of exon 7 of the ABO locus. Each such amplicon optionally can include additional sequence from intron 5,3′-UTR, or both intron 5 and 3′-UTR.

In certain embodiments, nucleotide sequences of the paired PCR amplification primers for ABO are

(sense)
(SEQ ID NO: 1)
5′-CCCTTTGCTTTCTCTGACTTGCG-3′;
and
(antisense)
(SEQ ID NO: 2)
5′-AGTTACTCACAACAGGACGGACA-3′.

The amplicons are fragmented to give a plurality of fragments about 200 to about 800 nucleotides long. In certain embodiments, the fragments are about 200 to about 500 nucleotides long. In certain embodiments, the fragments are about 300 to about 400 nucleotides long.

In certain embodiments, the method further comprises multiplexing with phase-defined genotyping of both alleles of at least one HLA locus of the subject.

Generally, the fragmentation will be random. In certain embodiments, the fragmentation comprises acoustical shearing, i.e., sonication. In certain embodiments, the fragmentation comprises enzymatic cleavage, for example using a transposase or the like. In certain embodiments, the fragmentation results in fragments having blunt ends. In certain embodiments, the fragmentation results in fragments having single-strand 5′ overhangs, 3′ overhangs, or both 5′ overhangs and 3′ overhangs. For example, fragmentation with acoustical shearing generally will result in fragments with single-strand 5′ overhangs, 3′ overhangs, or both 5′ overhangs and 3′ overhangs.

In certain embodiments, the method further includes end-repairing such fragments, for example with enzymes selected from T4 DNA polymerase, Klenow fragment of T4 DNA polymerase, T4 polynucleotide kinase, and any combination thereof.

In certain embodiments, the method further comprises labeling each fragment, prior to sequencing, with at least one source label. The source label can be designed and used to associate a source (subject or potential donor) with any given piece of DNA. For example, DNA from a subject can be amplified, sheared, optionally end-repaired, and optionally labeled, all prior to sequencing. Importantly, DNA from a first subject can be amplified, sheared, optionally end-repaired, and optionally labeled, all prior to pooling such DNA with corresponding DNA from a second subject, prior to sequencing. Advantageously, DNA from a first subject can be amplified, sheared, optionally end-repaired, and optionally labeled, all prior to pooling such DNA with corresponding DNA from a plurality of other subjects, prior to sequencing. In such embodiments, DNA of any one subject can be differentiated from DNA of any other subject or plurality of subjects, even when such DNA is pooled prior to sequencing.

In certain embodiments, the at least one source label is an oligonucleotide label. Such oligonucleotide label is sometimes referred to as a barcode or index, and it can be attached to an amplicon or fragment thereof by any suitable method, including, for example, ligation. Such oligonucleotide labels are generally synthetic oligonucleotides, about 8 to about 40 nucleotides long, characterized by a specific nucleotide sequence. In certain embodiments, an oligonucleotide label comprises about 15 to about 30 nucleotides. In certain embodiments, an oligonucleotide label comprises about 20 to about 25 nucleotides.

In certain embodiments, the oligonucleotide label is part of a longer oligonucleotide construct comprising additional functional sequence, e.g., annealing site or adapter suitable for making the modified fragment compatible with a sequencing primer, an immobilized bridge amplification primer of complementary sequence (part of the sequencing strategy), or both a sequencing primer and an immobilized bridge amplification primer.

In certain embodiments, each fragment is labeled with one source label.

In certain embodiments, each fragment is labeled with two source labels. The two source labels can be the same or different from one other.

For embodiments in which at least one source label is an oligonucleotide, generally such source label will be sequenced along with the amplified DNA to which it is attached.

In certain embodiments, the method further comprises attaching to each fragment, prior to sequencing, an oligonucleotide complementary to a sequencing primer.

In certain embodiments, the method further comprises attaching to each fragment, prior to sequencing, an oligonucleotide adapter complementary to at least one immobilized bridge amplification primer. Bridge amplification is part of and preparatory to sequencing-by-synthesis, whereby clusters of immobilized sequencing templates are formed on a surface. Each such cluster typically can include approximately 10⁶ copies of a given template.

The method optionally can include a clean-up step prior to sequencing. For example, the clean-up step can comprise a sizing step, a quantity normalization step, or both a sizing step and a quantity normalization step in preparation for sequencing.

In certain embodiments, the method is performed in a multiplex manner In certain embodiments, at least one HLA locus is co-amplified with the ABO locus. In certain embodiments, the method is performed in a multiplex manner such that the ABO locus amplicon is included with at least one HLA locus amplicon selected from the group consisting of HLA-A, HLA-B, HLA-C, HLA-DRB, HLA-DQB1, HLA-DPB1, HLA-DQA1, and HLA-DPA1, for preparation of a library for a given sample or for a given individual.

In certain embodiments, genomic DNA obtained from two or more subjects are analyzed in parallel. The number of subjects whose genomic DNA is analyzed in parallel can be as many as 10, 20, 50, 100, 200, or even more than 200.

Typically, the method comprises the step of pooling samples (amplicon fragments) prepared as described above from a plurality of loci and/or a plurality of subjects, prior to sequencing.

The fragments, e.g., pooled sample fragments, are then sequenced using next-generation sequencing, for example sequencing-by-synthesis, thereby generating a plurality of overlapping partial nucleotide sequences. Preferably, the sequencing will result in so-called deep sequencing. Sequencing depth refers to the total number of reads is many times larger than the length of the sequence under study. Coverage is the average number of reads representing a given nucleotide in the reconstructed sequence. Depth can be calculated from the length of the original genome or sequence under study (G), the number of reads (A1), and the average read length (L) as N×LIG. For example, a hypothetical genome or sequence with 2,000 base pairs reconstructed from 8 reads with an average length of 500 nucleotides will have 2× redundancy. The same hypothetical genome or sequence with 2,000 base pairs reconstructed from 80 reads with an average length of 500 nucleotides will have 20× redundancy, and the same hypothetical genome or sequence with 2,000 base pairs reconstructed from 400 reads with an average length of 500 nucleotides will have 100× redundancy. This parameter also enables one to estimate other quantities, such as the percentage of the genome covered by reads (sometimes also called coverage). A high coverage in shotgun sequencing is desired because it can overcome errors in base calling and assembly.

Result is many overlapping short reads that cover the area being sequenced. Confident single-nucleotide polymorphism (SNP) calls may typically require read depth of 100× but in some instances might require as little as 15×. Reads are “paired,” meaning sequence both sense and antisense. Software assembles sequence either de novo or compared to reference as scaffold.

The overlapping partial nucleotide sequences are then aligned to determine a contiguous composite nucleotide sequence encoding the majority of each allele of the ABO locus. This alignment step typically uses publicly or commercially available computer-based nucleotide sequence alignment tools, e.g., a genome browser.

In certain embodiments, the contiguous composite nucleotide sequence includes all of exon 6, all of intron 6, and all of exon 7. In certain such embodiments, the contiguous composite nucleotide sequence further includes at least a part of intron 5, at least a part of 3′-UTR, or at least a part of intron 5 and at least a part of 3′-UTR.

Following the alignment step just described, the method includes the step of comparing the contiguous composite nucleotide sequences to a library of reference genomic sequences encoding a region comprising exon 6 and exon 7 and the intervening intron of the ABO locus. This comparison step typically uses commercially available computer-based nucleotide sequence analysis tools and a user-defined library of known ABO genomic sequences, e.g., a subset of sequences available in GenBank.

Currently, ABO genomic sequences available in GenBank are poorly curated and difficult to use. An aspect of the present invention is the creation of an accurate and reliable ABO library of genomic sequences from GenBank entries. Additional ABO genomic sequences will be identified using the methods of the invention. Thus another aspect of the present invention is the creation of an accurate and reliable ABO library of genomic sequences from novel genomic sequences identified using the methods of the invention. Of course these various libraries can also be combined, so yet another aspect of the present invention is the creation of an accurate and reliable ABO library of genomic sequences from GenBank entries and from novel genomic sequences identified using the methods of the invention.

Similarly, ABO cDNA sequences currently available in GenBank are poorly curated and difficult to use. An aspect of the present invention is the creation of an accurate and reliable ABO library of cDNA sequences from GenBank entries. Additional ABO cDNA sequences will be identified using the methods of the invention. Thus another aspect of the present invention is the creation of an accurate and reliable ABO library of cDNA sequences from novel cDNA sequences identified using the methods of the invention. Of course these various libraries can also be combined, so that cDNA sequences and genomic sequences form the basis of an accurate and reliable ABO library useful for interpretation of sequencing results.

An aspect of the invention is the ability to identify the two subgroups of A, namely, A1 and A2. The invention can be used to type for A2 directly. As described above, the A2 “allele” arises from a single nucleotide (C) deletion in exon 7, giving rise to a frame-shift that extends the reading frame by 64 nucleotides (Yamamoto F et al., Biochem Biophys Res Commun 187:366-374, 1992) and encodes a glycosyltransferase with reduced activity compared to A (A1).

In certain embodiments, the method further includes the step of identifying each contiguous composite nucleotide sequence as either (i) a sequence encoding a known allele of the ABO locus, or (ii) a sequence encoding a novel allele of the ABO locus.

When NGS is used to obtain two phased sequences representing the maternal and paternal alleles of the ABO glycosyltransferase, software is used to compare the consensus allele sequences to a reference database of known allele sequences in order to predict the A, B, and O phenotypes of the individual. Since the ABO sequences are not curated and no ABO reference library is available, each sequence had to be obtained individually from GenBank and a reference library for sequence interpretation created. Currently a search of GenBank for human ABO sequences retrieves just over 900 sequences. Some of these sequences are duplicates, and some are only partial sequences of the glycosyltransferase gene. Some are listed in the National Center for Biotechnology Information (NCBI) database called dbRBC; many are not. Some are published; many are not. Out of the 900+ GenBank entries, sequences selected for the library were identified by us to represent common alleles as well as some rare alleles. FASTA sequences were retrieved from GenBank and trimmed to represent the same region of the gene represented in the ABO amplicon. A library of ABO sequences was created using Library Builder from Connexio Assign. Results from testing random individuals were used to identify other common alleles to add to the library so that the results from a majority of individuals could be interpreted as specific alleles.

Alleles included in the library on Feb. 25, 2016, are listed in Table 2.

TABLE 2

| dbRBC | | | | Phenotype from | | |
|---|---|---|---|---|---|---|
| ABO | dbRBC | Allele Name from | Name used | NCBI dbRBC or | dbRBC | |
| ID | Name | dbRBC and/or submitter | by Assign | submitter (noted) | Prevalence | GenBank ID |
| 83 | A301 | ABO-*A301 | A*0301/ | A3 | rare | AF134423- |
| | | | A3*0301 | | | 4424 (replaced |
| | | | | | | by AH007589) |
| 1457 | A101 | ABO*A101/A*101 | A*101 | A101 | common | AJ536122 |
| | abo1.02 | A*102 | A*102 | A | L/P | AB844268.1 |
| | | | | | variation | |
| | | | | | with 101, | |
| | | | | | Yi says | |
| | | | | | both are | |
| | | | | | common | |
| | | ABO*A201 | tbd | | | AJ536123.1 |
| 1265 | A201 | A2*01.01.2/A2*01012 | A2*01012 | A2 | | FN908802 |
| | var1 | | | | | |
| 1264 | A216 | A2.16.01.1/A2*16011 | A2*16011 | A2 | | FN908803 |
| 1277 | Bw26 | AB-weak/ABW*01 | BW*02 | B weak; B/O | rare | JF296309 |
| | | | | fusion | | |
| 103 | Ael01 | ABO*Ae101/AE*101 | AE*101 | Ael | rare | AJ536131 |
| 18 | B101 | ABO*B101/B*101 | B*101 | B | common | AF016622; |
| | | | | | | AJ536135 |
| 1254 | B101 | ABO*B1.01.1.3/B*10113 | B*10113 | B | | FN598478 |
| | var2 | | | | | |
| 24 | O01 | ABO*O01/O*01 | O*01 | O | common | AJ536140- |
| | | | | | | 6142 |
| 1262 | | ABO*O.01.01.5/O*01015 | O*01015 | O | looks | FN908801.1 |
| | | | | | common | |
| 27 | O02 | ABO*O02/O*02 | O*02 | O | | AJ536146- |
| | | | | | | 6147 |
| | | ABO*0.02.01.2/O*02.01.2 | O*2.01.2/2012 | O | | FN598480.1 |
| | | ABO-O02/O*0202 | O*0202 | O | | FJ851692.1 |
| 1761 | O68 | ABO*O.02.17.1/O*02171 | O*0217 | O | | FN908798.2 |
| 29 | O03 | ABO*O03/O*03 | O*03 | O | | AF440451; |
| | | | | | | AJ536152 |
| 33 | O06 | ABO*O06/O*06 | O*06 | O | | AJ536148 |
| | | ABO- | O*07tlse | O | | AF440459.1 |
| | | Ovar.tlse07/O*07tlse | | | | |
| 36 | O09 | | O*09tlse | O | | AF268885.2 |
| 39 | | nonfunctional ABO- | O*1102 | O | | AY138470.1 |
| | | O1V_G542A/O11/O*11 | | | | |
| 57 | | ABO*O26/O*26 | O*26 | O | | AJ536144.1 |
| 58 | | Ovar.tlse04/027/O*27 | O*27 | O | | AF440455.1 |
| 762 | | O54/O01bantu/O*54 | O*54 | O | rare | AY805749 |
| | | O59/ABO-O103.1/O*59 | O*59 | O | rare | AH014794.2 |
| 1452 | | aberrant O1 es | O*75 | O | rare | HF679090.1 |
| | | isolate/O75/O*75 | | | | |
| 101 | Aw08 | ABO*Aw08/OAW*08 | OAW*08 | Aw | rare | AJ536153.1 |
| | | allele | allele | | | |
| 1 | | A101.tlse16 | A*101tlse16 | | | AF448199.1 |
| | | ABOAV1S/ABO-Av1 | A2*1vYi | A2 | | AH008379.2 |
| | | ABO*Bwx | BO*1 weak | B weak | | KM068114.1 |
| | abo | ABO*O.01.13.1 | O*70 | O | | FN908804.1 |
| | abo | ABO-01v-B.tlse13 | O*41 | O | | AF440462.1 |
| 76 | | ABO-Ovar.tlse20 allele | tbd | | | AY138465.1 |

In certain embodiments, the method further includes assigning an ABO phenotype to the subject based on the phase-defined genotype of the ABO locus of the subject. For example, subjects found to have genotypes A/A or A/O are phenotyped as A; subjects found to have genotype A/B are phenotyped as AB; subjects found to have genotypes B/B or B/O are phenotyped as B; and subjects found to have genotype O/O are phenotyped as O. The A assignments can be either A1 or A2.

### Kits of the Invention

An aspect of the invention is a kit, comprising

(a) paired oligonucleotide polymerase chain reaction (PCR) amplification primers suitable for use to amplify, from a sample of human genomic DNA, DNA encoding the majority of the ABO glycosyltransferase gene of both alleles of at least one ABO locus;

(b) paired oligonucleotide adapters, each adapter oligonucleotide comprising a nucleotide sequence complementary to at least one bridge amplification primer immobilized on a substrate; and

(c) paired sequencing primers suitable for use to sequence amplification products prepared using the paired PCR amplification primers.

In certain embodiments, the DNA encoding the majority of the ABO glycosyltransferase gene of both alleles of at least one ABO locus is genomic DNA encoding a region comprising exon 6 and exon 7 of both alleles of the ABO locus. Such genomic DNA typically will include intron 6 and optionally can further include at least a portion of intron 5, at least a portion of the 3′-UTR, or both at least a portion of intron 5 and at least a portion of the 3′-UTR.

In certain embodiments, the kit further includes paired oligonucleotide PCR amplification primers suitable for use to amplify, from the sample of human genomic DNA, DNA encoding both alleles of at least one human leukocyte antigen (HLA) locus.

In certain embodiments, the at least one HLA locus is selected from the group consisting of HLA-A, HLA-B, and HLA-C.

In certain embodiments, the at least one HLA locus is selected from the group consisting of HLA-DRB, HLA-DQB1, HLA-DPB1, HLA-DQA1, and HLA-DPA1.

In certain embodiments, the paired PCR amplification primers for ABO are

(sense)
(SEQ ID NO: 1)
5′-CCCTTTGCTTTCTCTGACTTGCG-3′
and
(antisense)
(SEQ ID NO: 2)
5′-AGTTACTCACAACAGGACGGACA-3′.

In certain embodiments, the kit further comprises at least one enzyme selected from the group consisting of T4 DNA polymerase, Klenow fragment of T4 DNA polymerase, and T4 polynucleotide kinase; and at least one buffer suitable for activity of said T4 DNA polymerase, Klenow fragment of T4 DNA polymerase, and/or T4 polynucleotide kinase in repairing DNA fragments generated by shearing, e.g., acoustical shearing.

In certain embodiments, the kit further comprises a DNA polymerase and dATP in a buffer suitable for activity of said DNA polymerase to allow for adapter ligation.

In certain embodiments, the kit further comprises at least one source label.

In certain embodiments, the at least one source label is an oligonucleotide label.

In certain embodiments, the kit further comprises an oligonucleotide complementary to at least one of the paired sequencing primers.

Having now described the present invention in detail, the same will be more clearly understood by reference to the following examples, which are included herewith for purposes of illustration only and are not intended to be limiting of the invention.

### EXAMPLES

### Example 1

### Assignments of ABO out of 304 Samples Tested in Parallel With Serology

TABLE 3

| | | | | | NGS |
|---|---|---|---|---|---|
| | Blood Type | NGS | NGS | NGS | predicted |
| ID | Serology | ABO_1 | ABO_2 | Genotype | phenotype |
| 1 | A | A*102 | O*26 | A + O | A (A1) |
| 2 | A | A2*16011 | O*1015 | A2 + O | A (A2) |
| 3 | AB | A*101 | B*101 | A + B | AB |
| 4 | O | O*202 | O*202 | O + O | O |
| 5 | B | B*101 | O*54 | B + O | B |
| 6 | A | A*102 | O*1015 | A + O | A (A1) |
| 7 | B | B*101 | O*202 | B + O | B |
| 8 | O | O*1015 | O*1015 | O + O | O |

### Example 2

### Summary of Assignments of ABO by NGS (n=304 Samples).

TABLE 4

| | Number typed by NGS |
|---|---|
| Phenotype Assigned | |
| A | 102 |
| B | 44 |
| O | 142 |
| AB | 16 |
| Genotype Assigned | |
| A + A | 10 |
| A + O | 92 |
| B + B | 2 |
| B + O | 42 |
| O + O | 142 |
| A + B | 16 |
| Subgroups of A | |
| A1 | 105 |
| A2 | 23 |

### Example 3

TABLE 5
List of all six discrepancies of the NGS result and the previous serologic typing result
out of 304 samples tested. Of the 6 discrepancies, 3 were resolved in favor of NGS and
3 remain to be attributed. Samples were not available to retest by serology.

| | Serologic | | | NGS Predicted | NGS | | |
|---|---|---|---|---|---|---|---|
| NGS | Assignment | NGS | NGS | Low Resolution | Predicted | Discrepancy | |
| Run | Blood Type* | Allele 1 | Allele2 | Genotype | Phenotype | Explanation | ID |
| 116 | A corrected | O*02 | O*02 | O + O | O | discrepancy; | 413 |
| | to O | | | | | serology typing | |
| | | | | | | incorrectly entered by | |
| | | | | | | volunteer, O is correct | |
| 120 | B corrected | O*202 | O*202 | O + O | O | discrepancy; transcription | 186 |
| | to O | | | | | error in serologic entry, | |
| | | | | | | correct typing O | |
| 120 | AB corrected | A*101 | O*03 | A + O | A | discrepancy; volunteer | 327 |
| | to A | | | | | not sure if serology | |
| | | | | | | was A or AB so NGS | |
| | | | | | | likely correct | |
| 120 | AB | A*101 | O*09tlse | A + O | A | discrepancy; volunteer | 533 |
| | | | | | | confirms serology | |
| | | | | | | was AB | |
| 120 | O | A*101 | O*02 | A + O | A | discrepancy; unable to | 541 |
| | | | | | | recontact volunteer | |
| 117 | O | A*101 | A*101 | A + A | A | discrepancy | 74 |

*NGS assignments were compared to previous serologic ABO assignments. Out of the total 304 samples tested in parallel, 71 of the serologic results had been reported by transplant center during testing of patient and their hematopoietic progenitor cell donor. The remainder (n = 233) of the serologic results were reported by a volunteer based on their knowledge of their own blood group.

### Example 4

### Consensus Sequence of ABO From Sample Typed as A*102+O*26

The heterozygous deletion at position 612 (Connexio Assign MPS numbering) is underlined. The deletion at 612 is found commonly in O alleles. [Note: While the analysis software is able to phase nucleotides and identify genotypes, it is not yet able to produce a phased output, so a consensus sequence is shown.]

(SEQ ID NO: 5)
TCTCTTGTTTCCTGTCCCTTTGTTCTCCAAAGCCCCTGCAAAGGCCTGAT
AGGTACCTCCTACCTGGGGAGGGGCAGCGGGGGTTGGGTGCTGGGGAGGG
TTTGTTCCTATCTCTTTGCCAGCAAAGCTCAGCTTGCTGTGTGTTCCCRC
AGGTCCAATGTTGAGGGAGGGCTGGGAATGATTTGCCCGGTTGGAGTCGC
ATTTGCCTCTGGTTGGTTTCCCGGGGAAGGGCGGCTGCCTCTGGAAGGGT
GGTCAGAGGAGGCAGAAGCTGAGTGGAGTTTCCAGGTGGGGGCGGCCGTG
TGCCAGAGGCGCATGTGGGTGGCACCCTGCCAGCTCCATGTGACCGCACG
CCTCTCTCCATGTGCAGTAGGAAGGATGTCCTCGTGGTgACCCCTTGGCT
GGCTCCCATTGTCTGGGAGGGCACATTCAACATCGACATCCTCAACGAGC
AGTTCAGGCTCCAGAACACCACCATTGGGTTAACTGTGTTTGCCATCAAG
AAGTAAGTCAGTGAGGTGGCCGAGGGTAGAGACCCAGGCAGTGGCGAGTG
ACTGTGGACATTGAGGTCTCTCCTTGTGTTCAAGACAGAGTGGGGTGGCG
GCCAGCCTTGTCCTCCCAGAGGGTAGATGGGAAAGGTCATTCATGCAGCA
TCTTACTGAGCTCATGTGGGCTCGTGGGCTCGTGGGCTCGCCAGGTCGGT
AAAACCCAGCTCCTTCTCCAGAGGCTGCGTCTCACCCAGGGATGGTGGCT
TCTGCTGCCCCCTCCTCTCTGTAACTGTGGCCGGCCGTCATGCTGAGCCA
CCCCCTCAATACAAGGCTCCAGATGTTTCCTGCTCACTGACCAGAGATAG
CAGGAGGGGGACACCTGTTTGCTGTCCTTGGACCCTAGAAAGAGGATGCT
GGCAGAGCCGTGGTCACTTCTCTGTCAGATGTAGGTGGGGCAGGCAAAGC
AGTTGGCCCCAGACACCAAAGGAAGTGGCTGACCCACAAGGCCCTGGGAC
TCTGGGCCAGGCCAGAGAGGGAGCTAGCCAGGCAACCGCAGACACATACT
TGACTTCTCGGCAGCTGTGGGCAGCTGGGCCAGCGACAGTGGCGGAGGCC
AGGAATGACTTACTCTTAGGAATAGGTGCAGTTCAAGCCTGGAGGGAGGA
AGCTCTAGGGTGCAGAGGCGGGTGTGTGGAGGCCTCGCGTGCAGCTTATA
ATGAGGGAGCACGTGGCCGGCCTGGCCATAAGAGGGGCAGCTGCGTGGGG
AGGCGTGGCTCAGGCCAGGCTGAGGGGGAGTGAGCRGACGCCAGCCTGCG
GCCTGCTACCAGCCTCCAGCCACCTGCCCTCAGCCCTCCTTAGTAAGAGG
GGGTGCTGGTGGTCCCCCATCGCTGGGAAGAGGATGAAGTGAATCGCAGC
CCGAGGACTCGCTCAGGACAGGGCAGGAGAACGTGGTGCATCTGCTGCTC
TAAGCCTTCCAATGGCCGCTGGCGGGCGGGTGCAGGACGGGCCTCCTGCA
GCCCAGGGGTGCACGGCCGGCGGCTCCCCCAGCCCCCGTCCGCCTGCCTT
GCAGATACGTGGCTTTCCTGAAGCTGTTCCTGGAGACGGCGGAGAAGCAC
TTCATGGTGGGCCACCGTGTCCACTACTATGTCTTCACCGACCAGCYGGC
CGCGGTGCCCCGCGTGACGCTGGGGACCGGTCGGCAGCTGTCAGTGCTGG
AGGTGCGCGCCTACAAGCGCTGGCAGGACGTGTCCATGCGCCGCATGGAG
ATGATCAGTGACTTCTGCGAGCGGCGCTTCCTCAGCGAGGTGGATTACCT
GGTGTGCGTGGACGTGGACATGGAGTTCCGCGACCACGTGGGCGTGGAGA
TCCTGACTCCGCTGTTCGGCACCCTGCACCCCGGCTTCTACGGAAGCAGC
CGGGAGGCCTTCACCTACGAGCGCCGGCCCCAGTCCCAGGCCTACATMCC
CAAGGACGAGGGCGATTTCTACTACCTGGGGGGGTTCTTCGGGGGGTCGG
TGCAAGAGGTGCAGCGGCTCACCAGGGCCTGCCACCAGGCCATGATGGTC
GACCAGGCCAACGGCATCGAGGCCGTGTGGCACGACGAGAGCCACCTGAA
CAAGTACCTGCTGCGCCACAAACCCACCAAGGTGCTCTCCCCCGAGTACT
TGTGGGACCAGCAGCTGCTGGGCTGGCCCGCCGTCCTGAGGAAGCTGAGG
TTCACTGCGGTGCCCAAGAACCACCAGGCGGTCCGGAACCCGTGAGCGGC
TGCCAGGGGCTCTGGGAGGGCTGCCGGCAGCCCCGTCCCCCTCCCGCCCT
TGGTTTTAG

Example 5

### Consensus Sequence of Sample Typed as A2*1012+O*01

The heterozygous deletion at position 612 (indicated by “g”) (Connexio Assign MPS numbering) is underlined, as is the heterozygous deletion at 2464 (indicated by “c”). The deletion at 612 is found commonly in O alleles. The deletion at 2464 is found in the subgroup of A called A2. [Note: While the analysis software is able to phase nucleotides and identify genotypes, it is not yet able to produce a phased output, so a consensus sequence is shown.]

(SEQ ID NO: 6)
TCTCTTGTTTCCTGTCCCTTTGTTCTCCAAAGCCCCTGCAAAGGCCTGAT
AGGTACCTCCTACCTGGGGAGGGGCAGCGGGGGTTGGGTGCTGGGGAGGG
TTTGTTCCTATCTCTTTGCCAGCAAAGCTCAGCTTGCTGTGTGTTCCCRC
AGGTCCAATGTTGAGGGAGGGCTGGGAATGATTTGCCCGGTTGGAGTCGC
ATTTGCCTCTGGTTGGTTTCCCGGGGAAGGGCGGCTGCCTCTGGAAGGGT
GGTCAGAGGAGGCAGAAGCTGAGTGGAGTTTCCAGGTGGGGGCGGCCGTG
TGCCAGAGGCGCATGTGGGTGGCACCCTGCCAGCTCCATGTGACCGCACG
CCTCTCTCCATGTGCAGTAGGAAGGATGTCCTCGTGGTgACCCCTTGGCT
GGCTCCCATTGTCTGGGAGGGCACATTCAACATCGACATCCTCAACGAGC
AGTTCAGGCTCCAGAACACCACCATTGGGTTAACTGTGTTTGCCATCAAG
AAGTAAGTCAGTGAGGTGGCCGAGGGTAGAGACCCAGGCAGTGGCGAGTG
ACTGTGGACATTGAGGTCTCTCCTTGTGTTCAAGACAGAGTGGGGTGGCG
GCCAGCCTTGTCCTCCCAGAGGGTAGATGGGAAAGGTCATTCATGCAGCA
TCTTACTGAGCTCATGTGGGCTCGTGGGCTCGTGGGCTCGCCAGGTCGGT
AAAACCCAGCTCCTTCTCCAGAGGCTGCGTCTCACCCAGGGATGGTGGCT
TCTGCTGCCCCCTCCTCTCTGTAACTGTGGCCGGCCGTCATGCTGAGCCA
CCCCCTCAATACAAGGCTCCAGATGTTTCCTGCTCACTGACCAGAGATAG
CAGGAGGGGGACACCTGTTTGCTGTCCTTGGACCCTAGAAAGAGGATGCT
GGCAGAGCCGTGGTCACTTCTCTGTCAGATGTAGGTGGGGCAGGCAAAGC
AGTTGGCCCCAGACACCAAAGGAAGTGGCTGACCCACAAGGCCCTGGGAC
TCTGGGCCAGGCCAGAGAGGGAGCTAGCCAGGCAACCGCAGACACATACT
TGACTTCTCGGCAGCTGTGGGCAGCTGGGCCAGCGACAGTGGCGGAGGCC
AGGAATGACTTACTCTTAGGAATAGGTGCAGTTCAAGCCTGGAGGGAGGA
AGCTCTAGGGTGCAGAGGCGGGTGTGTGGAGGCCTCGCGTGCAGCTTATA
ATGAGGGAGCACGTGGCCGGCCTGGCCATAAGAGGGGCAGCTGCGTGGGG
AGGCGTGGCTCAGGCCAGGCTGAGGGGGAGTGAGCRGACGCCAGCCTGCG
GCCTGCTACCAGCCTCCAGCCACCTGCCCTCAGCCCTCCTTAGTAAGAGG
GGGTGCTGGTGGTCCCCCATCGCTGGGAAGAGGATGAAGTGAATCGCAGC
CCGAGGACTCGCTCAGGACAGGGCAGGAGAACGTGGTGCATCTGCTGCTC
TAAGCCTTCCAATGGCCGCTGGCGGGCGGGTGCAGGACGGGCCTCCTGCA
GCCCAGGGGTGCACGGCCGGCGGCTCCCCCAGCCCCCGTCCGCCTGCCTT
GCAGATACGTGGCTTTCCTGAAGCTGTTCCTGGAGACGGCGGAGAAGCAC
TTCATGGTGGGCCACCGTGTCCACTACTATGTCTTCACCGACCAGCYGGC
CGCGGTGCCCCGCGTGACGCTGGGGACCGGTCGGCAGCTGTCAGTGCTGG
AGGTGCGCGCCTACAAGCGCTGGCAGGACGTGTCCATGCGCCGCATGGAG
ATGATCAGTGACTTCTGCGAGCGGCGCTTCCTCAGCGAGGTGGATTACCT
GGTGTGCGTGGACGTGGACATGGAGTTCCGCGACCACGTGGGCGTGGAGA
TCCTGACTCCGCTGTTCGGCACCCTGCACCCCGGCTTCTACGGAAGCAGC
CGGGAGGCCTTCACCTACGAGCGCCGGCCCCAGTCCCAGGCCTACATCCC
CAAGGACGAGGGCGATTTCTACTACCTGGGGGGGTTCTTCGGGGGGTCGG
TGCAAGAGGTGCAGCGGCTCACCAGGGCCTGCCACCAGGCCATGATGGTC
GACCAGGCCAACGGCATCGAGGCCGTGTGGCACGACGAGAGCCACCTGAA
CAAGTACCTGCTGCGCCACAAACCCACCAAGGTGCTCTCCCCCGAGTACT
TGTGGGACCAGCAGCTGCTGGGCTGGCCCGCCGTCCTGAGGAAGCTGAGG
TTCACTGCGGTGCCCAAGAACCACCAGGCGGTCCGGAACCcGTGAGCGGC
TGCCAGGGGCTCTGGGAGGGCTGCCGGCAGCCCCGTCCCCCTCCCGCCCT
TGGTTTTAG

### Example 6

### Consensus Sequence for Sample Typed as A*101+B*101

Variation in exon 7 at Assign MPS position 2199 (M, i.e., C or A) and 2206 (S, i.e., G or C). [Note: While the analysis software is able to phase nucleotides and identify genotypes, it is not yet able to produce a phased output, so a consensus sequence is shown.]

(SEQ ID NO: 7)
TCTCTTGTTTCCTGTCCCTTTGTTCTCCAAAGCCCCTGCAAAGGCCTGAT
AGGTACCTCCTACCTGGGGAGGGGCAGCGGGGGTTGGGTGCTGGGGAGGG
TTTGTTCCTATCTCTTTGCCAGCAAAGCTCAGCTTGCTGTGTGTTCCCGC
AGGTCCAATGTTGAGGGAGGGCTGGGAATGATTTGCCCGGTTGGAGTCGC
ATTTGCCTCTGGTTGGTTTCCCGGGGAAGGGCGGCTGCCTCTGGAAGGGT
GGTCAGAGGAGGCAGAAGCTGAGTGGAGTTTCCAGGTGGGGGCGGCCGTG
TGCCAGAGGCGCATGTGGGTGGCACCCTGCCAGCTCCATGTGRCCGCACG
CCTCTCTCCATGTGCAGTAGGAAGGATGTCCTCGTGGTGACCCCTTGGCT
GGCTCCCATTGTCTGGGAGGGCACRTTCAACATCGACATCCTCAACGAGC
AGTTCAGGCTCCAGAACACCACCATTGGGTTAACTGTGTTTGCCATCAAG
AAGTAAGTCAGTGAGGTGGCCGAGGGTAGAGACCCAGGCAGTGKCGAGTG
ACTGTGGACATTGAGGTCTCTCCTTGTGTTCAAGACAGAGTGGGGTGGCG
GCCAGCCTTGTCCTCCCAGAGGGTAGATGGGAAAGGTCATTCATGCAGCA
TCTTACTGAGCTCAYGTGGGCTCGTGGGCTYGTGGGCTCGCCAGGTCGGT
AAAACCCAGCTCCTTCTCCAGAGGCTGCGTCTCACCCAGGGATGGTGGCT
TCTGCTGCCCCCTCCTCTCTGTRACTGTGGCYGGCCGTCATGCTGAGCCA
CCCCCTCAATACAAGGCTCCAGATGTTTCCTGCTCACTGACCAGAGATAG
CAGGAGGGGGACACCTGTTTGCTGTCCTTGGACCCTAGAAAGAGGATGCT
GGCAGAGCCGTGGTCACTTCTCTGTCAGATGTAGGTGGGGCAGGCAARGC
AGTTGGCCCCAGACACCAAAGGAAGTGGCTGACCCACAAGGCCCTGGGAC
TCTGGGCCAGGCCAGAGAGGGAGCTAGCCAGGCAACCGCAGACACATACT
TGACTTCTCGGCAGCTGTGGGCAGCTGGGCCAGCGACAGTGGCGGAGGCC
AGGAATGACTTACTCTTAGGAATAGGTGCRGTTCAAGCCTGGAGGGAGGA
AGCTCTAGGGTGCAGAGGCGGGTGTGTGGAGGCCTCGCGTGCAGCTTATA
ATGAGGGAGCACGTGGCCGGCCTGGCCATAAGAGGGGCAGCTGCGTGGGG
AGGCGTGGCTCAGGCCAGGCTGAGGGGGAGTGAGCGGRCGCCAGCCTGCG
GCCTGCTACCAGCCTCCAGCCACCTGCCCTCAGCCCTCCTTAGTAAGAGG
GGGTGCTGGTGGTCCCCCATCGCTGGGAAGAGGATGAAGTGARTCGCAGC
CCRAGGACTCGCTCAGGACAGGGCAGGAGAACGTGGTGCATCTGCTGCTC
TRAGCCTTCCAATGGCCGCTGGCGGGCGGGTGCAGGACGGGCCTCCTGCA
GCCCAGGGGTGCACGGCCGGCGGCTCCCCCAGCCCCCGTCCGCCTGCCTT
GCAGATACGTGGCTTTCCTGAAGCTGTTCCTGGAGACGGCGGAGAAGCAC
TTCATGGTGGGCCACCGTGTCCACTACTATGTCTTCACCGACCAGCCGGC
CGCGGTGCCCCGCGTGACGCTGGGGACCGGTCGGCAGCTGTCAGTGCTGG
AGGTGSGCGCCTACAAGCGCTGGCAGGACGTGTCCATGCGCCGCATGGAG
ATGATCAGTGACTTCTGCGAGCGGCGCTTCCTCAGCGAGGTGGATTACCT
GGTGTGCGTGGACGTGGACATGGAGTTCCGCGACCAYGTGGGCGTGGAGA
TCCTGACTCCGCTGTTCGGCACCCTGCACCCCRGCTTCTACGGAAGCAGC
CGGGAGGCCTTCACCTACGAGCGCCGGCCCCAGTCCCAGGCCTACATCCC
CAAGGACGAGGGCGATTTCTACTACMTGGGGGSGTTCTTCGGGGGGTCGG
TGCAAGAGGTGCAGCGGCTCACCAGGGCCTGCCACCAGGCCATGATGGTC
GACCAGGCCAACGGCATCGAGGCCGTGTGGCACGACGAGAGCCACCTGAA
CAAGTACCTRCTGCGCCACAAACCCACCAAGGTGCTCTCCCCCGAGTACT
TGTGGGACCAGCAGCTGCTGGGCTGGCCCGCCGTCCTGAGGAAGCTGAGG
TTCACTGCGGTGCCCAAGAACCACCAGGCGGTCCGGAACCCGTGAGCGGC
TGCCAGGGGCTCTGGGAGGGCTGCCRGCAGCCCCGTCCCCCTCCCGCCCT
TGGTTTTAG

### Example 7

### Consensus Nucleotide Sequence of Sample Typed O*02+O*02

The homozygous deletion at position 612 (Connexio Assign MPS numbering) is underlined. The deletion at 612 is found commonly in O alleles. [Note: While the analysis software is able to phase nucleotides and identify genotypes, it is not yet able to produce a phased output, so a consensus sequence is shown.]

(SEQ ID NO: 8)
TCTCTTGTTTCCTGTCCCTTTGTTCTCCAAAGCCCCTGCAAAGGCCTGAT
AGGTACCTCCTACCTGGGGAGGGGCAGCGGGGGTTGGGTGCTGGGGAGGG
TTTGTTCCTATCTCTTTGTCAGCAAAGCTCAGCTTGCTGTGTGTTCCCGC
AGGTCCAATGTTGAGGGAGGGCTGGGAATGATTTGCCCGGTTGGAGTCGC
ATTTGCCTCTGGTTGGTTTCCCGGGGAAGGGCGGCTGCCTCTGGAAGGGT
GGTCAGAGGAGGAAGAAGCTGAGTGGAGTTTCCAGGTGGGGGCGGCCGTG
TGCCAGAGGCGCATGTGGGTGGCACCCTGCCAGCTCCATATGACCGCACG
CCTCTCTCCATGTGCAGTAGGAAGGATGTCCTCGTGGTACCCCTTGGCTG
GCTCCCATTGTCTGGGAGGGCACGTTCAACATCGACATCCTCAACGAGCA
GTTCAGGCTCCAGAACACCACCATTGGGTTAACTGTGTTTGCCATCAAGA
AGTAAGTCAGTGAGGTGGCCGAGGGTAGAGACCCAGGCAGTGGCGAGTGA
CTGTGGACATTGAGGTCTCTCCTTGTGTTCAAGACAGAGAGGGGTGGCGG
CCAGCCTTGTCCTCCCAGAGGGTAGATGGGAAAGGTCATTCATGCAGCAT
CTTACTGAGCTCACGTGGGCTCGTGGGCTCGTGGGCTCACCAGGTCGGTA
AAACCCAGCTCCTTCTCCAGAGGCTGTGTCTCACCGAGGGATGGTGGCTT
CTGCTGCCCCCTCCTCTCTGTAACTGTGGCCGGCCGTCATGCTGAGCCAC
CCCCTCAATACAAGGCTCCAGATGTTTCCTGCTCACTGACCAGAGATAGC
AGGAGGGGGACACCTGTTTGCTGTCCTTGGACCCTAGAAAGAGGATGCTG
GCAGAGCCGTGGTCACTTCTCTGTCAGATGTAGGTGGGGCAGGCAAGGCA
GTTGGCCCCAGACACCAAAGGAAGTGGCTGACCCACAAGGCCCCGGGACT
CTGGGCCAGGCCAGAGAGGGAGCTAGCCAGGCAACCGCAGACACATACTT
GACTTCTCGGCAGCTGTGGGCAGCTGGGCCAGCGACAGTGGCGGAGGCCA
GGAATGACTTACTCTTAGGAATAGGTGCAGTTCAAGCCTGGAGGGAGGAA
GCTCTAGGGTGCAGAGGCGGGTGTGTGGAGGCCTCGCGTGCAGCTTATAA
TGAGGGAGCACGTGGCCAGCCTGGCCATAAGAGGGGCAGCTGCGTGGGGA
GGCGTGGCTCAGGCCAGGCTGAGGGGGAGTGAGCGGGCGCCAGCCTGCGG
CCTGCTACCAGCCTCCAGCCACCTGCCCTCAGCCCTCCTTAGTAAGAGGG
GGTGCTGGTGGTCCCCCATCGCTGGGAAGAGGATGAAGTGAGTCGCAGCC
CGAGGACTCGCTCAGGACAGGGCAGGAGAACGTGGTGCATCTGCTGCTCT
GAGCCTTCCAATGGCCGCTGGCGGGCGGGTGCAGGACGGGCCTCCTGCAG
CCCAGGGGTGCGCAGCCGGCGGCTCCCCCAGCCCCCGTCCGCCTGCCTTG
CAGATACGTGGCTTTCCTGAAGCTGTTCCTGGAGACGGCGGAGAAGCACT
TCATGGTGGGCCACCGTGTCCACTACTATGTCTTCACCGACCAGCCGGCC
GCGGTGCCCCGCGTGACGCTGGGGACCGGTCGGCAGCTGTCAGTGCTGGA
GGTGCGCGCCTACAAGCGCTGGCAGGACGTGTCCATGCGCCGCATGGAGA
TGATCAGTGACTTCTGCGAGCGGCGCTTCCTCAGCGAGGTGGATTACCTG
GTGTGCGTGGACGTGGACATGGAGATCCGCGACCACGTGGGCGTGGAGAT
CCTGACTCCACTGTTCGGCACCCTGCACCCCGGCTTCTACGGAAGCAGCC
GGGAGGCCTTCACCTACGAGCGCCGGCCCCAGTCCCAGGCCTACATCCCT
AAGGACGAGGGCGATTTCTACTACCTGGGGGGGTTCTTCGGGGGGTCGGT
GCAAGAGATGCAGCGGCTCACCAGGGCCTGCCACCAGGCCATGATGGTCG
ACCAGGCCAACGGCATCGAGGCCGTGTGGCACGACGAGAGCCACCTGAAC
AAGTACCTGCTGCGCCACAAACCCACCAAGGTGCTCTCCCCCGAGTACTT
GTGGGACCAGCAGCTGCTGGGCTGGCCCGCCGTCCTGAGGAAGCTGAGGT
TCACTGCGGTGCCCAAGAACCACCAGGCGGTCCGGAACCCGTGAGCGGCT
GCCAGGGGCTCTGGGAGGGCTGCCGGCAGCCCCGTCCCCCTCCCGCCCTT
GGTTTTAG

### INCORPORATION BY REFERENCE

All patents and published patent applications mentioned in the description above are incorporated by reference herein in their entirety.

### EQUIVALENTS

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

1. A method of phase-defined genotyping of both alleles of the glycosyltransferase (ABO) locus of a subject, comprising
amplifying a sample of human genomic DNA encoding a region comprising exon 6 and exon 7 of both alleles of the ABO locus, thereby forming a plurality of amplicons;
fragmenting the amplicons to give a plurality of fragments about 200 to about 800 nucleotides long;
sequencing the fragments using next-generation sequencing, thereby generating a plurality of overlapping partial nucleotide sequences;
aligning the overlapping partial nucleotide sequences to determine a contiguous composite nucleotide sequence encoding a region comprising exon 6 and exon 7 of each allele of the ABO locus;
comparing the contiguous composite nucleotide sequences to a library of reference genomic sequences encoding a region comprising exon 6 and exon 7 of the ABO locus; and
identifying each contiguous composite nucleotide sequence as either (i) a sequence encoding a region comprising a known exon 6 and exon 7 of the ABO locus, or (ii) a sequence encoding a region comprising a novel exon 6 and/or exon 7 of the ABO locus.

2. A method of phase-defined genotyping of both alleles of the glycosyltransferase (ABO) locus of a subject, comprising
amplifying a sample of human genomic DNA encoding a region comprising exon 6 and exon 7 of both alleles of the ABO locus, thereby forming a plurality of amplicons;
fragmenting the amplicons to give a plurality of fragments about 200 to about 800 nucleotides long;
sequencing the fragments using sequencing-by-synthesis, thereby generating a plurality of overlapping partial nucleotide sequences;
aligning the overlapping partial nucleotide sequences to determine a contiguous composite nucleotide sequence encoding a region comprising exon 6 and exon 7 of each allele of the ABO locus;
comparing the contiguous composite nucleotide sequences to a library of reference genomic sequences encoding a region comprising exon 6 and exon 7 of the ABO locus; and
identifying each contiguous composite nucleotide sequence as either (i) a sequence encoding a region comprising a known exon 6 and exon 7 of the ABO locus, or (ii) a sequence encoding a region comprising a novel exon 6 and/or exon 7 of the ABO locus.

3. The method of claim 1, wherein each amplicon comprises DNA encoding exon 6, intron 6, and exon 7 of the ABO locus.

4. The method of claim 1, wherein the fragments are about 200 to about 500 nucleotides long.

5. The method of claim 4, wherein the fragments are about 300 to about 400 nucleotides long.

6. The method of claim 1, further comprising multiplexing with phase-defined genotyping of both alleles of at least one human leukocyte antigen (HLA) locus of the subject.

7. The method of claim 1, wherein the fragmenting comprises acoustical shearing.

8. The method of claim 1, further comprising end-repairing the fragments.

9. The method of claim 1, further comprising labeling each fragment, prior to sequencing, with at least one source label.

10. The method of claim 9, wherein the at least one source label is an oligonucleotide label.

11. The method of claim 9, wherein each fragment is labeled with one source label.

12. The method of claim 9, wherein each fragment is labeled with two source labels.

13. The method of claim 9, further comprising sequencing the at least one source label.

14. The method of claim 1, further comprising attaching to each fragment, prior to sequencing, an oligonucleotide complementary to a sequencing primer.

15. The method of claim 1, further comprising attaching to each fragment, prior to sequencing, an oligonucleotide adapter complementary to at least one immobilized bridge amplification primer.

16. The method of claim 1, wherein the method is performed in a multiplex manner.

17. The method of claim 1, further comprising assigning an ABO phenotype to the subject based on the phase-defined genotype of the ABO locus of the subject.

18. A kit, comprising
(a) paired oligonucleotide polymerase chain reaction (PCR) amplification primers suitable for use to amplify, from a sample of human genomic DNA, DNA encoding both alleles of the glycosyltransferase (ABO) locus;
(b) paired oligonucleotide adapters, each oligonucleotide adapter comprising a nucleotide sequence complementary to at least one bridge amplification primer immobilized on a substrate; and
(c) paired sequencing primers suitable for use to sequence amplification products prepared using the paired PCR amplification primers.

19. The kit of claim 18, further comprising
(d) paired oligonucleotide PCR amplification primers suitable for use to amplify, from the sample of human genomic DNA, DNA encoding both alleles of at least one human leukocyte antigen (HLA) locus.

20. The kit of claim 19, wherein the at least one HLA locus is selected from the group consisting of HLA-A, HLA-B, and HLA-C.

21-27. (canceled)


SEQUENCE LISTING (35 sequences)

SEQ ID NO: 1 - DNA, 23 bp, Artificial Sequence
  Description of Artificial Sequence Synthetic oligonucleotide
ccctttgctttctctgacttgcg

SEQ ID NO: 2 - DNA, 23 bp, Artificial Sequence
  Description of Artificial Sequence Synthetic oligonucleotide
agttactcacaacaggacggaca

SEQ ID NO: 3 - DNA, 11 bp, Homo sapiens
cctgggggggt

SEQ ID NO: 4 - DNA, 11 bp, Homo sapiens
catgggggcgt

SEQ ID NO: 5 - DNA, 2309 bp, Homo sapiens
tctcttgtttcctgtccctttgttctccaaagcccctgcaaaggcctgataggtacctcctacctggggaggggcagcgggggttgggtgctggggagggtttgttcctatctctttgccagcaaagctcagcttgctgtgtgttcccrcaggtccaatgttgagggagggctgggaatgatttgcccggttggagtcgcatttgcctctggttggtttcccggggaagggcggctgcctctggaagggtggtcagaggaggcagaagctgagtggagtttccaggtgggggcggccgtgtgccagaggcgcatgtgggtggcaccctgccagctccatgtgaccgcacgcctctctccatgtgcagtaggaaggatgtcctcgtggtgaccccttggctggctcccattgtctgggagggcacattcaacatcgacatcctcaacgagcagttcaggctccagaacaccaccattgggttaactgtgtttgccatcaagaagtaagtcagtgaggtggccgagggtagagacccaggcagtggcgagtgactgtggacattgaggtctctccttgtgttcaagacagagtggggtggcggccagccttgtcctcccagagggtagatgggaaaggtcattcatgcagcatcttactgagctcatgtgggctcgtgggctcgtgggctcgccaggtcggtaaaacccagctccttctccagaggctgcgtctcacccagggatggtggcttctgctgccccctcctctctgtaactgtggccggccgtcatgctgagccaccccctcaatacaaggctccagatgtttcctgctcactgaccagagatagcaggagggggacacctgtttgctgtccttggaccctagaaagaggatgctggcagagccgtggtcacttctctgtcagatgtaggtggggcaggcaaagcagttggccccagacaccaaaggaagtggctgacccacaaggccctgggactctgggccaggccagagagggagctagccaggcaaccgcagacacatacttgacttctcggcagctgtgggcagctgggccagcgacagtggcggaggccaggaatgacttactcttaggaataggtgcagttcaagcctggagggaggaagctctagggtgcagaggcgggtgtgtggaggcctcgcgtgcagcttataatgagggagcacgtggccggcctggccataagaggggcagctgcgtggggaggcgtggctcaggccaggctgagggggagtgagcrgacgccagcctgcggcctgctaccagcctccagccacctgccctcagccctccttagtaagagggggtgctggtggtcccccatcgctgggaagaggatgaagtgaatcgcagcccgaggactcgctcaggacagggcaggagaacgtggtgcatctgctgctctaagccttccaatggccgctggcgggcgggtgcaggacgggcctcctgcagcccaggggtgcacggccggcggctcccccagcccccgtccgcctgccttgcagatacgtggctttcctgaagctgttcctggagacggcggagaagcacttcatggtgggccaccgtgtccactactatgtcttcaccgaccagcyggccgcggtgccccgcgtgacgctggggaccggtcggcagctgtcagtgctggaggtgcgcgcctacaagcgctggcaggacgtgtccatgcgccgcatggagatgatcagtgacttctgcgagcggcgcttcctcagcgaggtggattacctggtgtgcgtggacgtggacatggagttccgcgaccacgtgggcgtggagatcctgactccgctgttcggcaccctgcaccccggcttctacggaagcagccgggaggccttcacctacgagcgccggccccagtcccaggcctacatmcccaaggacgagggcgatttctactacctgggggggttcttcggggggtcggtgcaagaggtgcagcggctcaccagggcctgccaccaggccatgatggtcgaccaggccaacggcatcgaggccgtgtggcacgacgagagccacctgaacaagtacctgctgcgccacaaacccaccaaggtgctctcccccgagtacttgtgggaccagcagctgctgggctggcccgccgtcctgaggaagctgaggttcactgcggtgcccaagaaccaccaggcggtccggaacccgtgagcggctgccaggggctctgggagggctgccggcagccccgtccccctcccgcccttggttttag

SEQ ID NO: 6 - DNA, 2309 bp, Homo sapiens
tctcttgtttcctgtccctttgttctccaaagcccctgcaaaggcctgataggtacctcctacctggggaggggcagcgggggttgggtgctggggagggtttgttcctatctctttgccagcaaagctcagcttgctgtgtgttcccrcaggtccaatgttgagggagggctgggaatgatttgcccggttggagtcgcatttgcctctggttggtttcccggggaagggcggctgcctctggaagggtggtcagaggaggcagaagctgagtggagtttccaggtgggggcggccgtgtgccagaggcgcatgtgggtggcaccctgccagctccatgtgaccgcacgcctctctccatgtgcagtaggaaggatgtcctcgtggtgaccccttggctggctcccattgtctgggagggcacattcaacatcgacatcctcaacgagcagttcaggctccagaacaccaccattgggttaactgtgtttgccatcaagaagtaagtcagtgaggtggccgagggtagagacccaggcagtggcgagtgactgtggacattgaggtctctccttgtgttcaagacagagtggggtggcggccagccttgtcctcccagagggtagatgggaaaggtcattcatgcagcatcttactgagctcatgtgggctcgtgggctcgtgggctcgccaggtcggtaaaacccagctccttctccagaggctgcgtctcacccagggatggtggcttctgctgccccctcctctctgtaactgtggccggccgtcatgctgagccaccccctcaatacaaggctccagatgtttcctgctcactgaccagagatagcaggagggggacacctgtttgctgtccttggaccctagaaagaggatgctggcagagccgtggtcacttctctgtcagatgtaggtggggcaggcaaagcagttggccccagacaccaaaggaagtggctgacccacaaggccctgggactctgggccaggccagagagggagctagccaggcaaccgcagacacatacttgacttctcggcagctgtgggcagctgggccagcgacagtggcggaggccaggaatgacttactcttaggaataggtgcagttcaagcctggagggaggaagctctagggtgcagaggcgggtgtgtggaggcctcgcgtgcagcttataatgagggagcacgtggccggcctggccataagaggggcagctgcgtggggaggcgtggctcaggccaggctgagggggagtgagcrgacgccagcctgcggcctgctaccagcctccagccacctgccctcagccctccttagtaagagggggtgctggtggtcccccatcgctgggaagaggatgaagtgaatcgcagcccgaggactcgctcaggacagggcaggagaacgtggtgcatctgctgctctaagccttccaatggccgctggcgggcgggtgcaggacgggcctcctgcagcccaggggtgcacggccggcggctcccccagcccccgtccgcctgccttgcagatacgtggctttcctgaagctgttcctggagacggcggagaagcacttcatggtgggccaccgtgtccactactatgtcttcaccgaccagcyggccgcggtgccccgcgtgacgctggggaccggtcggcagctgtcagtgctggaggtgcgcgcctacaagcgctggcaggacgtgtccatgcgccgcatggagatgatcagtgacttctgcgagcggcgcttcctcagcgaggtggattacctggtgtgcgtggacgtggacatggagttccgcgaccacgtgggcgtggagatcctgactccgctgttcggcaccctgcaccccggcttctacggaagcagccgggaggccttcacctacgagcgccggccccagtcccaggcctacatccccaaggacgagggcgatttctactacctgggggggttcttcggggggtcggtgcaagaggtgcagcggctcaccagggcctgccaccaggccatgatggtcgaccaggccaacggcatcgaggccgtgtggcacgacgagagccacctgaacaagtacctgctgcgccacaaacccaccaaggtgctctcccccgagtacttgtgggaccagcagctgctgggctggcccgccgtcctgaggaagctgaggttcactgcggtgcccaagaaccaccaggcggtccggaacccgtgagcggctgccaggggctctgggagggctgccggcagccccgtccccctcccgcccttggttttag

SEQ ID NO: 7 - DNA, 2309 bp, Homo sapiens
tctcttgtttcctgtccctttgttctccaaagcccctgcaaaggcctgataggtacctcctacctggggaggggcagcgggggttgggtgctggggagggtttgttcctatctctttgccagcaaagctcagcttgctgtgtgttcccgcaggtccaatgttgagggagggctgggaatgatttgcccggttggagtcgcatttgcctctggttggtttcccggggaagggcggctgcctctggaagggtggtcagaggaggcagaagctgagtggagtttccaggtgggggcggccgtgtgccagaggcgcatgtgggtggcaccctgccagctccatgtgrccgcacgcctctctccatgtgcagtaggaaggatgtcctcgtggtgaccccttggctggctcccattgtctgggagggcacrttcaacatcgacatcctcaacgagcagttcaggctccagaacaccaccattgggttaactgtgtttgccatcaagaagtaagtcagtgaggtggccgagggtagagacccaggcagtgkcgagtgactgtggacattgaggtctctccttgtgttcaagacagagtggggtggcggccagccttgtcctcccagagggtagatgggaaaggtcattcatgcagcatcttactgagctcaygtgggctcgtgggctygtgggctcgccaggtcggtaaaacccagctccttctccagaggctgcgtctcacccagggatggtggcttctgctgccccctcctctctgtractgtggcyggccgtcatgctgagccaccccctcaatacaaggctccagatgtttcctgctcactgaccagagatagcaggagggggacacctgtttgctgtccttggaccctagaaagaggatgctggcagagccgtggtcacttctctgtcagatgtaggtggggcaggcaargcagttggccccagacaccaaaggaagtggctgacccacaaggccctgggactctgggccaggccagagagggagctagccaggcaaccgcagacacatacttgacttctcggcagctgtgggcagctgggccagcgacagtggcggaggccaggaatgacttactcttaggaataggtgcrgttcaagcctggagggaggaagctctagggtgcagaggcgggtgtgtggaggcctcgcgtgcagcttataatgagggagcacgtggccggcctggccataagaggggcagctgcgtggggaggcgtggctcaggccaggctgagggggagtgagcggrcgccagcctgcggcctgctaccagcctccagccacctgccctcagccctccttagtaagagggggtgctggtggtcccccatcgctgggaagaggatgaagtgartcgcagcccraggactcgctcaggacagggcaggagaacgtggtgcatctgctgctctragccttccaatggccgctggcgggcgggtgcaggacgggcctcctgcagcccaggggtgcacggccggcggctcccccagcccccgtccgcctgccttgcagatacgtggctttcctgaagctgttcctggagacggcggagaagcacttcatggtgggccaccgtgtccactactatgtcttcaccgaccagccggccgcggtgccccgcgtgacgctggggaccggtcggcagctgtcagtgctggaggtgsgcgcctacaagcgctggcaggacgtgtccatgcgccgcatggagatgatcagtgacttctgcgagcggcgcttcctcagcgaggtggattacctggtgtgcgtggacgtggacatggagttccgcgaccaygtgggcgtggagatcctgactccgctgttcggcaccctgcaccccrgcttctacggaagcagccgggaggccttcacctacgagcgccggccccagtcccaggcctacatccccaaggacgagggcgatttctactacmtgggggsgttcttcggggggtcggtgcaagaggtgcagcggctcaccagggcctgccaccaggccatgatggtcgaccaggccaacggcatcgaggccgtgtggcacgacgagagccacctgaacaagtacctrctgcgccacaaacccaccaaggtgctctcccccgagtacttgtgggaccagcagctgctgggctggcccgccgtcctgaggaagctgaggttcactgcggtgcccaagaaccaccaggcggtccggaacccgtgagcggctgccaggggctctgggagggctgccrgcagccccgtccccctcccgcccttggttttag

SEQ ID NO: 8 - DNA, 2308 bp, Homo sapiens
tctcttgtttcctgtccctttgttctccaaagcccctgcaaaggcctgataggtacctcctacctggggaggggcagcgggggttgggtgctggggagggtttgttcctatctctttgtcagcaaagctcagcttgctgtgtgttcccgcaggtccaatgttgagggagggctgggaatgatttgcccggttggagtcgcatttgcctctggttggtttcccggggaagggcggctgcctctggaagggtggtcagaggaggaagaagctgagtggagtttccaggtgggggcggccgtgtgccagaggcgcatgtgggtggcaccctgccagctccatatgaccgcacgcctctctccatgtgcagtaggaaggatgtcctcgtggtaccccttggctggctcccattgtctgggagggcacgttcaacatcgacatcctcaacgagcagttcaggctccagaacaccaccattgggttaactgtgtttgccatcaagaagtaagtcagtgaggtggccgagggtagagacccaggcagtggcgagtgactgtggacattgaggtctctccttgtgttcaagacagagaggggtggcggccagccttgtcctcccagagggtagatgggaaaggtcattcatgcagcatcttactgagctcacgtgggctcgtgggctcgtgggctcaccaggtcggtaaaacccagctccttctccagaggctgtgtctcaccgagggatggtggcttctgctgccccctcctctctgtaactgtggccggccgtcatgctgagccaccccctcaatacaaggctccagatgtttcctgctcactgaccagagatagcaggagggggacacctgtttgctgtccttggaccctagaaagaggatgctggcagagccgtggtcacttctctgtcagatgtaggtggggcaggcaaggcagttggccccagacaccaaaggaagtggctgacccacaaggccccgggactctgggccaggccagagagggagctagccaggcaaccgcagacacatacttgacttctcggcagctgtgggcagctgggccagcgacagtggcggaggccaggaatgacttactcttaggaataggtgcagttcaagcctggagggaggaagctctagggtgcagaggcgggtgtgtggaggcctcgcgtgcagcttataatgagggagcacgtggccagcctggccataagaggggcagctgcgtggggaggcgtggctcaggccaggctgagggggagtgagcgggcgccagcctgcggcctgctaccagcctccagccacctgccctcagccctccttagtaagagggggtgctggtggtcccccatcgctgggaagaggatgaagtgagtcgcagcccgaggactcgctcaggacagggcaggagaacgtggtgcatctgctgctctgagccttccaatggccgctggcgggcgggtgcaggacgggcctcctgcagcccaggggtgcgcagccggcggctcccccagcccccgtccgcctgccttgcagatacgtggctttcctgaagctgttcctggagacggcggagaagcacttcatggtgggccaccgtgtccactactatgtcttcaccgaccagccggccgcggtgccccgcgtgacgctggggaccggtcggcagctgtcagtgctggaggtgcgcgcctacaagcgctggcaggacgtgtccatgcgccgcatggagatgatcagtgacttctgcgagcggcgcttcctcagcgaggtggattacctggtgtgcgtggacgtggacatggagatccgcgaccacgtgggcgtggagatcctgactccactgttcggcaccctgcaccccggcttctacggaagcagccgggaggccttcacctacgagcgccggccccagtcccaggcctacatccctaaggacgagggcgatttctactacctgggggggttcttcggggggtcggtgcaagagatgcagcggctcaccagggcctgccaccaggccatgatggtcgaccaggccaacggcatcgaggccgtgtggcacgacgagagccacctgaacaagtacctgctgcgccacaaacccaccaaggtgctctcccccgagtacttgtgggaccagcagctgctgggctggcccgccgtcctgaggaagctgaggttcactgcggtgcccaagaaccaccaggcggtccggaacccgtgagcggctgccaggggctctgggagggctgccggcagccccgtccccctcccgcccttggttttag

SEQ ID NO: 9 - protein, 13 aa, Homo sapiens
GDFYYMGAFFGGS

SEQ ID NO: 10 - protein, 13 aa, Homo sapiens
GDFYYLGGFFGGS

SEQ ID NO: 11 - protein, 34 aa, Homo sapiens
LRCPRTTRRSGTRERLPGALGGLPAAPSPSRPWF

SEQ ID NO: 12 - DNA, 103 bp, Homo sapiens
ctgcggtgcccaagaaccaccaggcggtccggaacccgtgagcggctgccaggggctctgggagggctgccagcagccccgtccccctcccgcccttggtttt

SEQ ID NO: 13 - DNA, 102 bp, Homo sapiens
ctgcggtgcccaagaaccaccaggcggtccggaaccgtgagcggctgccaggggctctgggagggctgccagcagccccgtccccctcccgcccttggtttt

SEQ ID NO: 14 - DNA, 102 bp, Homo sapiens
ctgcggtgccaaagaaccaccaggcggtccggaaccgtgagcggctgccaggggctctgggagggctgccagcagccccgtccccctcccgcccttggtttt

SEQ ID NO: 15 - DNA, 102 bp, Homo sapiens
ctgcggtgcccaagaaccaccaggcggcccggaacccgtgagcggctgccaggggctctgggagggctgccagcagccccgtcccctcccgcccttggtttt

SEQ ID NO: 16 - DNA, 102 bp, Homo sapiens
ctgcggtgcccaagagccaccaggcggtccggaaccgtgagcggctgccaggggctctgggagggctgccagcagccccgtccccctcccgcccttggtttt

SEQ ID NO: 17 - DNA, 102 bp, Homo sapiens
ctgcggtgcccaagaaccaccaggcggtccggacccgtgagcggctgccaggggctctgggagggctgccagcagccccgtccccctcccgcccttggtttt

SEQ ID NO: 18 - DNA, 102 bp, Homo sapiens
ctgcggtgcccaagaaccaccaggcggtctggaaccgtgagcggctgccaggggctctgggagggctgccagcagccccgtccccctcccgcccttggtttt

SEQ ID NO: 19 - DNA, 102 bp, Homo sapiens
ctgcggtgcccaagaaccaccaggcggtccggaacccgtgagcggctgccaggggctctgggagggctgccagcagcccgtccccctcccgcccttggtttt

SEQ ID NO: 20 - DNA, 102 bp, Homo sapiens
cttcggtgcccaagaaccaccaggcggtccggaaccgtaagcggctgccaggggctctgggagggctgccagcagccccgtccccctcccgcccttggtttt

SEQ ID NO: 21 - DNA, 102 bp, Homo sapiens
ctgcggtgcccaagaaccaccaggcggtccggaaccgcgagcggctgccaggggctctgggagggctgccagcagcctcgtccccctcccgcccttggtttt

SEQ ID NO: 22 - DNA, 102 bp, Homo sapiens
ctgcggtgcccaagaacccccaggcggtccggaaccgtgagcggctgccaggggctctgggagggctgccagcagccccggccccctcccgcccttggtttt

SEQ ID NO: 23 - DNA, 102 bp, Homo sapiens
ctgcggtgcccaagaaccaccaggcggtccggaaccgtgagcggctgccaggggctctgggatggctgccagcagccccgtccccctcccgccctttgtttt

SEQ ID NO: 24 - DNA, 97 bp, Homo sapiens
ctcgtggtgaccccttggctggctcccattgtctgggagggcacrttcaacatcgacatcctcaaygagcagttcaggctccagaacaccaccattg

SEQ ID NO: 25 - DNA, 97 bp, Homo sapiens
ctcgtggtgaccccttggctggctcccattgtctgggagggcactttcaacatcgacatcctcaacgagcagttcaggctccagaacaccaccattg

SEQ ID NO: 26 - DNA, 18 bp, Homo sapiens
ctcgtggtgaccccttgg

SEQ ID NO: 27 - DNA, 17 bp, Homo sapiens
ctcgtggtaccccttgg

SEQ ID NO: 28 - DNA, 10 bp, Homo sapiens
gtggtgaccc

SEQ ID NO: 29 - DNA, 9 bp, Homo sapiens
gtggtaccc

SEQ ID NO: 30 - DNA, 14 bp, Homo sapiens
ggaaccskkragcg

SEQ ID NO: 31 - DNA, 14 bp, Homo sapiens
ggaacccgtgagcg

SEQ ID NO: 32 - DNA, 15 bp, Homo sapiens
tacmtggggrsgttc

SEQ ID NO: 33 - DNA, 15 bp, Homo sapiens
tacctggggrggttc

SEQ ID NO: 34 - DNA, 15 bp, Homo sapiens
tacatggggrcgttc

SEQ ID NO: 35 - DNA, 15 bp, Homo sapiens
tacmtgggggsgttc